(19) United States
(12) Patent Application Publication
(10) Pub. No.: US 2008/0269121 A1
(43) Pub. Date: Oct. 30, 2008

(54) Methods and Compositions Involving A1c Subunit of L-Type Calcium Channels in Smooth Muscle Cells

(75) Inventor: Sushil K. Sarna, Houston, TX (US)

Correspondence Address: FULBRIGHT & JAWORSKI L.L.P., 600 CONGRESS AVE., SUITE 2400, AUSTIN, TX, 78701, US

(73) Assignee: The Board of Regents of the University of Texas System

(21) Appl. No.: 11/817,220

(22) PCT Filed: Feb. 24, 2006
(86) PCT No.: PCT/US06/06560

§ 371 Date: Apr. 7, 2008

Related U.S. Application Data
(60) Provisional application No. 60/656,231, filed on Feb. 25, 2005.

(51) Int. Cl. A61K 38/16; A61K 31/135; A61P 1/00

(52) U.S. Cl. 514/12; 514/653

(57) ABSTRACT

The present invention concerns methods and composition for modulating the level of gene expression of the α_{1C} subunit of the L-type calcium channel. Such methods and compositions provide preventative and therapeutic benefits with respect to gastrointestinal motility disorders. In examples of the invention, sustained changes in gene expression of the α_{1C} subunit are effected by a low dose of a modulator given over an extended period of time. This provides methods and compositions for inducing sustained contractility or relaxation of gut smooth muscle cells.

This application claims priority to U.S. Provisional Application Ser. No. 60/656,231, filed Feb. 25, 2005, which is incorporated herein by reference in its entirety.

The government may own rights in the present invention pursuant to grant numbers DK32346 and DK072414 from the National Institutes of Health.

### BACKGROUND OF THE INVENTION

I. Field of the Invention

The present application generally concerns molecular biology, biochemistry, physiology, and internal medicine. More particularly it relates to methods and compositions for mediating relaxation, inhibiting contraction, or contraction of the gastrointestinal tract through a genomic effect on the α_{1C} subunit of the L-type calcium channels in gut smooth muscle cells.

II. Description of Related Art

The enteric nervous system plays a critical role in the regulation of gastrointestinal motility function (Wood, 2004). The excitatory and inhibitory motor neurons of the myenteric plexus release neurotransmitters to contract or relax smooth muscle cells. The putative neurotransmitters of the excitatory motor neurons are acetylcholine (ACh) and substance P (SP), while those of the inhibitory neurons are adenosine triphosphate (ATP), vasoactive intestinal polypeptide (VIP)/pituitary adenylate cyclase-activating peptide (PACAP) and nitric oxide (NO).

The general understanding is that there is a continuous basal release of inhibitory neurotransmitters to keep the smooth muscle cells in the resting state. For example, the addition of tetrodotoxin, which blocks sodium channel enteric neural conduction of action potentials, whether to the muscle bath or by close intra-arterial administration in intact animals immediately stimulates contractions that are thought to be due to the blockade of the continuous basal release of the inhibitory neurotransmitters (Hou et al., 1989; Wood, 1972). The non-genomic effects of the inhibitory neurotransmitters in the regulation of smooth muscle relaxation—the pharmacological effects—have been studied extensively. It is well established that VIP/PACAP, on binding to their receptors on smooth muscle cells, activate adenylyl cyclase to produce cyclic 3′-5′ adenosine monophosphate (cAMP) that mediates smooth muscle relaxation.

cAMP is also a well known mediator of gene expression through the cAMP response element (CRE) on the promoters of its target genes (Montminy et al., 1986). The transcription factor CRE binding protein (CREB) binds to this element. Thus far, CREB has been reported to regulate well over 100 genes, most of which relate to proliferation, differentiation and growth function of cells (Silva et al., 1998; Lee and Masson, 1993; Lonze and Ginty, 2002; Shaywitz and Greenberg, 1999).

To date, however, there has been no direct evidence regarding the regulation of gut smooth muscle cell contractility on a genomic, as opposed to pharmacological, basis. The pharmacological regulation of gut smooth muscle cell contractility as a result of a burst of neurotransmitter release is well known to occur immediately and their effects are short-lived. Moreover, these pharmacological effects of a particular neurotransmitter may be different from its nonpharmacological effects. For example, any sustained pharmacological effect as a result of VIP would paralyze the gut. This is demonstrated by U.S. Pat. No. 5,681,816, entitled “Method of Inducing Temporary Paralysis of the Gastrointestinal Tract During Medical Procedure.” In this patent, the “effect of VIP in reducing gastrointestinal motility is almost immediate upon being administered to a patient” (col. 5, lines 35-37). The timing of the effect (“almost immediate”) indicates VIP was exerting a pharmacological, as opposed to genomic, effect. Thus, with the neurotransmitter VIP, a burst of VIP causes inhibition of contractility of smooth muscle cells. This type of burst can disrupt the homeostasis of gut smooth muscle cell contractility. Diseases and conditions also disrupt the homeostasis of contractility.

Thus, far, the use of VIP or other neurotransmitters has not been considered in as a treatment modality based on its genomic effects. Furthermore, there continues to be a need for treatments that address disruptions of homeostasis in gut smooth muscle cell contractility.

Moreover, in certain situations this disruption is related to an inflammatory response. The muscularis propria is richly endowed with immunocytes (Kalff et al., 1998; Eskandari et al., 1997; Cicalese et al., 1996; Eskandari et al., 1998), whose function is to protect the smooth muscle cells and enteric neurons from injury. This immune system is primed to respond readily to inflammatory signals as a result of mucosal inflammation or due to elevated circulating levels of pro-inflammatory cytokines in disease (Komatsu et al., 2001). However, homeostasis in smooth muscle function has to be maintained by a fine balance between the pro-inflammatory and anti-inflammatory factors. A disruption of this balance can lead to physiological disturbances, for example, gastrointestinal disorders. There is a need for therapies that address these disturbances by maintaining or re-establishing homeostasis.

### SUMMARY OF THE INVENTION

The present invention is based on scientific data that gene expression of the α_{1C} subunit of the L-type calcium channel can be altered, leading to long-term alteration in the contractility of gut smooth muscle cells. Furthermore, the data indicate that VIP/PACAP may also be anti-inflammatory neuropeptides that counter the initiation of the signaling cascade that activates the transcription factor NF-κB, the activation of NFκB resulting in the suppression of cell contractility during inflammation (Shi et al., 2003). “Long-term” means at least about 30 minutes or more.

Accordingly, the present invention concerns modulating the expression of the α_{1C} subunit of the L-type calcium channel, so as to modulate the amount of α_{1C} subunit in a cell. Therefore, the invention is directed toward preventative and therapeutic compositions and methods involving gastrointestinal motility disorders in which an alteration in the contractility of gut smooth muscle cells, depending on the symptoms exhibited by the afflicted patient, will effect a physiological benefit.

In particular embodiments, the present invention involves methods for treating a gastrointestinal motility disorder in a subject comprising administering, delivering, and/or contacting (either directly or indirectly) an effective amount of an α_{1C} modulator (including VIP and/or PACAP or antagonist thereof) to/with a subject's gut smooth muscle cells, whereby the modulator modulates the long-term expression of α_{1C} polypeptide in the cells and modifies the contractility of the gut. It will be understood that administration to the subject refers to administration of an exogenous α_{1C} modulator (i.e., modulator that was not previously within the subject's body at the concentration and/or for time period of administration). The term “treating” includes ameliorating or curing the disease, condition, or disorder; retarding the rate or extent of the progression of a disease, condition, or disorder; and, reducing the time span of, the occurrence of, or the extent of any discomfort and/or pain; and/or physical limitations associated with recuperation from a disease, disorder or condition.

Other methods include treating diarrhea comprising administering to a subject with diarrhea or at risk for diarrhea an effective amount of an α_{1C} repressor. The α_{1C} repressor will prevent contractility and/or promote relaxation (including inhibiting contraction) of gut smooth muscle cells so as to restore homeostasis and/or prevent or alleviate diarrhea. Of note is the underlying contractile state of the gut related to diarrhea. Diarrhea is typically the result of a hypercontractility of the gut, however in some circumstances the contractility of the gut may be suppressed in such a manner that transit of gastrointestinal tract is increased. On some occasions hypocontractility may result in diarrhea. A physician treating for diarrhea will be able to determine the etiology of the diarrhea and treat a subject accordingly.

In certain embodiments, the repressor will promote sustained relaxation or inhibition of contraction of gut smooth muscle cells. “Sustained relaxation” or “inhibition of contraction” refers to relaxation or a reduction in the ability to contract, respectively, of gut smooth cells for at least 0.5, 1, 2 hours or more. It is contemplated that in some embodiments of the invention, relaxation or inhibition of contractility of the gut is maintained for about, at least about, or at most about 30 minutes, and/or 1, 2, 3, 4, 5, 6, 7, 8, 9, 10, 11, 12, 13, 14, 15, 16, 17, 18, 19, 20, 21, 22, 23, 24 hours or more, 1, 2, 3, 4, 5, 6, 7 days, and/or 1, 2, 3, 4, 5 weeks, and/or 1, 2, 3, 4, 5, 6, 7, 8, 9, 10, 11, 12 or more months, or any range derivable therein.

The present invention also covers methods for treating constipation comprising administering to a subject with constipation an effective amount of an α_{1C} inducer (including VIP and/or PACAP). The α_{1C} inducer will induce sustained contractility of the gut smooth muscle cells or sustained sensitization of the gut smooth muscle to contractility (ability to contract in response to a contractile stimulus) so as to restore homeostasis and/or prevent or alleviate constipation.

In certain embodiments an inducer will induce sustained contractility of gut smooth muscle cells or sustained sensitization of the gut smooth muscle to contractility. “Sustained contractility” refers to contractility of gut smooth cells or to an ability to respond to contractile stimuli for at least 0.5, 1, 2 hours or more. It is contemplated that in some embodiments of the invention, contractility of the gut is maintained for about, at least about, or at most about 30 minutes, and/or 1, 2, 3, 4, 5, 6, 7, 8, 9, 10, 11, 12, 13, 14, 15, 16, 17, 18, 19, 20, 21, 22, 23, 24 hours or more, 1, 2, 3, 4, 5, 6, 7 days, and/or 1, 2, 3, 4, 5 weeks, and/or 1, 2, 3, 4, 5, 6, 7, 8, 9, 10, 11, 12 or more months, or any range derivable therein. Contractility of the gut refers to RPCs, GMCs, TC, or various combinations thereof. Contractility in general relates to proper coordination of the three types of contractions in the gut.

In some embodiments, the invention concerns methods for stimulating contractility of gut smooth muscle cells in a subject comprising administering to the subject an amount of an α_{1C} inducer (including VIP and/or PACAP) to increase the expression of α_{1C} polypeptide in the cells. In some cases, expression of α_{1C} polypeptide is significantly increased for 24 hours or more. The expression of the α_{1C} polypeptide can be increased for at least 1, 2, 3, 4, 5, or more days.

Other aspects of the invention include methods for stimulating sustained contractility of gut smooth muscle cells in a subject comprising administering to the subject an effective amount of VIP, PACAP, norepinephrine, or a VIP or PACAP receptor agonist, whereby the effective amount increases expression of α_{1C} polypeptide in the cells. In some embodiments, contractility of the gut smooth muscle cells had been suppressed or was suspected of being suppressed by an inflammatory cytokine. Examples of an inflammatory cytokine include, but are not limited to, TNFα.

There are additional methods for suppressing contractility of gut smooth muscle cells in a subject comprising administering to the subject an effective amount of an α_{1C} repressor, whereby the effective amount represses the expression of α_{1C} polypeptide in the cells. In certain aspects a repressor would be administered to a subject identified or suspected of having diarrhea (typically the result of hypercontractility). In contrast, constipation is typically the result of hypocontractility.

Another method is for preventing or treating suppression of contractility (hypocontractility), e.g., from exposure to an inflammatory cytokine, in gut smooth muscle cells in a subject comprising administering to the subject an effective amount of an α_{1C} inducer, whereby the effective amount inhibits NFκB repression of α_{1C} expression. In certain embodiments, the α_{1C} inducer inhibits one or more subunits of NFκB.

Other aspects of the invention relate to a method for inducing sustained contractility, relaxation, or inhibition of contractility of smooth muscle cells in a subject comprising administering to the subject a relatively low dose of an α_{1C} modulator, whereby the modulator modulates the long-term expression of α_{1C} polypeptide in the cells. In some embodiments, the smooth muscle cells are in the gastrointestinal tract, while in others they are in the vasculature of the heart.

The term “effective amount” refers to the amount in a course of therapy that achieves a particular result. The term “modulates” refers to altering the amount of α_{1C} polypeptide in a cell by at least about 5, 10, 20, 30% or more relative to the amount prior to administration of exogenous α_{1C} modulator to the subject or a cell. It is contemplated that “modulates” includes an increase or a decrease in the amount of α_{1C} polypeptide of about, at least about, or at most about 5, 10, 15, 20, 25, 30, 35, 40, 45, 50, 60, 70, 80, 90, 100, 110, 120, 130, 140, 150, 200, 250, 300, 250, 400% or more, or any range derivable therein. The term “long-term expression” refers to the amount of α_{1C} polypeptide in a cell over a period of time of at least 2, 12, 24 hours or more. It is contemplated that a subject can be any animal, including mammals, such as humans.

In certain embodiments, the long-term expression of an α_{1C} subunit is significantly increased, while in others it is significantly decreased. The term “significantly” refers to an increase or decrease in α_{1C} subunit polypeptide amount that is at least 10, 20, 30, 40, 50% or more different than prior to administration of exogenous α_{1C} modulator.

An “α_{1C} modulator” will be understood to refer to a compound or substance that alters the amount of α_{1C} subunit of the L-type calcium channel protein in a cell by affecting the level of α_{1C} gene expression (genomic effect instead of a pharmacologic effect). Any Embodiment of the invention directed to an α_{1C} modulator shall be understood to include specific implementations using VIP or PACAP as a modulator of α_{1C}, as well as antagonist of VIP or PACAP. An α_{1C} modulator may reduce the amount of α_{1C} subunit polypeptide by inhibiting transcription of the α_{1C} gene or reducing or eliminating induction of transcription of the α_{1C} gene α_{1C} repressor); alternatively, a different α_{1C} modulator may increase the amount of α_{1C} subunit polypeptide by inducing transcription of the α_{1C} gene or inhibiting a repressor of transcription of the α_{1C} gene (α_{1C} inducer). The skilled artisan will know which type of modulator to employ depending on the subject's symptoms related to a gastrointestinal motility disorder. It may be that only a single type of modulator is needed for a particular patient, but that in another patient a combination of both types of modulator are needed. In the latter case, it is contemplated that one type may be administered for a period of time and thereafter the other type is administered for a period of time. This treatment regimen, as well as any other treatment regimen, may be repeated as needed.

It is particularly contemplated that modulation of α_{1C} transcription is modulation of the α1C1B promoter. An α_{1C} modulator may include a protein (including VIP or PACAP), nucleic acid, or small molecule. It may be wholly or partially synthetic, recombinant, or purified from a natural source. Furthermore, in any embodiment discussed with respect to VIP or PACAP, it is contemplated that the other neuropeptide may be implemented in a similar manner as that embodiment. The same is contemplated for any other α_{1C} modulator, if appropriate.

Moreover, multiple α_{1C} modulators may be administered, such as 1, 2, 3, 4, 5, 6 or more different kinds of α_{1C} modulators to a subject. It is contemplated that multiple modulators may be administered either at the same time (for example, as a cocktail) or at different times.

In certain embodiments, the α_{1C} modulator is a substance that affects how much CREB binds to the promoter of the α_{1C} gene. In particular cases, the α_{1C} modulator is VIP, PACAP or norepinephrine. Both PACAP-38 and PACAP-27 are contemplated, unless otherwise specified. These modulators act as α_{1C} inducers. Moreover, the present invention includes α_{1C} inducers that are agonists of VIP, PACAP, or norepinephrine receptors, as well as variants of VIP, PACAP, or norepinephrine that function like the native molecules. Additional α_{1C} inducers include modulators that inhibit repressors of α_{1C} gene expression. NFκB is a repressor of α_{1C} gene expression. NP↓B is induced by TNF-α. Consequently, the present invention is also directed at agents that inhibit NFκB. In some embodiments, an α_{1C} inducer is a molecule that inhibits one or more subunits of NFκB, such as p50 and p65. In certain embodiments, the α_{1C} inducer is an NFκB inhibitor, for example, an siRNA directed to the p50 and/or p65 subunits of NFκB.

In other circumstances the α_{1C} modulator is an α_{1C} repressor. Embodiments employ antagonists of VIP, PACAP, or norepinephrine receptors as α_{1C} repressors. In certain embodiments, a VIP receptor antagonist is VIP₁₀₋₂₈.

It is contemplated that the α_{1C} modulator is comprised in a pharmacologically or pharmaceutically acceptable formulation in most embodiments of the invention.

In some embodiments of the invention, the α_{1C} modulator is administered to the subject over a period of time between about 0.5, 1, 2 hours and 10, 20, 30 days. Over a period of time includes both continuous administration (e.g., infusion or time release) or repetitive administration of a low dose α_{1C} modulator. Administration of an agent for at least 0.5, 1, or 2 hours or more will be referred to as “extended.” It is specifically contemplated that the α_{1C} modulator is administered to the subject over all extended period of time of about, of at least about, or at most about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96 hours or more, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more, 1, 2, 3, 4, 5, 6, 7, 8, 9, 10, 11, 12 weeks or more, 1, 2, 3, 4 months or more, or any range derivable therein.

In other embodiments, methods involve administering a low dose or dosage of the α_{1C} modulator to the subject. It is contemplated that the dose is considered a low dose if the amount given is about or less than about 25 nM/kg/day. It will be understood that the amount given to the subject is dependent on the weight of the subject and it reflects the amount given in a day (i.e., a 24-hour period). In some embodiments, a subject is given about, less than about, or at most about 0.005, 0.05, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 100, 110, 120, 130, 140, 150 nM/kg/day, or any range derivable therein, so long as a genomic effect is being mediated. A sustained pharmacological effect will cause diarrhea. Alternatively, the amount of an α_{1C} modulator that is administered can be expressed in terms of nanogram (ng). In certain embodiments, the amount given is about, less than about, or at most about 0.005, 0.05, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 ng/kg/day, or any range derivable therein, so long as a genomic effect is being mediated. A sustained pharmacological effect will cause diarrhea. The amount given may be administered to the subject throughout or during an extended period of time (as opposed to a single administration given over the course of less than a minute or two). In this case, the amount given to the patient during the time period may be fairly constant or it may fluctuate, however, what is relevant is the total amount in a 24 hour period that is specified.

In other embodiments, the amount of an α_{1C} modulator may be expressed in other units. In some embodiments, a patient is given about, at least about or at most about 0.1, 0.2, 0.3, 0.4, 0.5, 0.6, 0.7, 0.8, 0.9, 1, 2, 3, 4, 5, 6, 7, 8, 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micromolar (μM)/minute, or any range derivable therein. This can be given by infusion or other systemic administration. Again, the effect to be achieved is a genomic effect through the administration of the modulator.

In certain embodiments, an effective amount of an α_{1C} modulator for the treatment of a gastrointestinal motility disorder is a low dose administered to the subject for an extended period of time. The low dose may be a particular dose and the period of time may be a particular period of time.

The present invention may involve an α_{1C} modulator formulated for time release or sustained release. Such formulations are well known to those of skill in the art.

In some embodiments, the α_{1C} modulator is formulated for administration to the subject transdermally, intravenously, orally, or intrarectally. It is further contemplated that the α_{1C} modulator may be formulated for administration using a patch or as a suppository.

Methods of the invention are contemplated for subjects with, exhibiting symptoms of, at risk for, suspected of or identified as having a gastrointestinal motility disorder. Subjects may have diarrhea (e.g., hypercontractility), constipation (e.g., hypocontractility), or a combination of both, or diarrhea and constipation alternately. A subject with diarrhea will be understood as a subject who has had diarrhea within 24 hours of being treated for the diarrhea by the recited methods. It is contemplated, of course, that the subject may have had diarrhea within an even shorter time span. A subject may be at risk for or identified as having such a disorder because of a variety of reasons including, but not limited to, identification of a microbial infection, intake of a drug or other agent that is known to have a side effect of a gastrointestinal motility disorder (for example, by causing diarrhea or constipation), genetic or familial susceptibility to a gastrointestinal motility disorder. Moreover, subjects may be diagnosed with or suspected of having irritable bowel syndrome, gastroparesis, or inflammatory bowel disease. Furthermore, the subject may be diagnosed with or suspected of having a gastrointestinal infection when methods of the invention are employed. In some embodiments of the invention, methods including identification of a subject in need of such treatment may involve taking a patient history, doing a patient interview, performing one or more tests on the subject, and/or diagnosing the patient with a gastrointestinal motility disorder.

α_{1C} modulators may be formulated and/or administered alone or in combination with a traditional therapy. In subjects suspected of having or diagnosed with a gastrointestinal infection, some embodiments of the invention also involve administering an antibiotic. Alternatively, an α_{1C} modulator may be formulated with and/or administered sequentially or simultaneously with a laxative, anti-diarrhea, or anti-inflammatory medication.

It is also contemplated that methods and compositions may be applied to smooth muscle cells other than gut smooth muscle cells. It is contemplated that the contractility of smooth muscle cells such as vascular smooth muscle cells (VSMCs) can be altered using an (cc modulator to provoke a genomic effect on contractility. It is contemplated that the embodiments discussed herein with respect to gut smooth muscle cells can be applied to other smooth muscle cells. In specific embodiments, VSMCs are targeted to provide a treatment for a vascular or cardiovascular disease or condition. In still further embodiments, there is a method for treating hypertension by providing an effective amount of an α_{1C} modulator to alter the contractility of a vascular smooth muscle cell.

The present invention also concerns pharmaceutical compositions comprising an α_{1C} modulator or other agent (e.g., forskolin and analogous agents) that increases cAMP in the muscle and/or smooth muscle of the gut. In some embodiments, the modulator is formulated for time-release or sustained (systemic) administration. The modulator may be an α_{1C} inducer or an α_{1C} repressor. Moreover, the composition may include one or more modulators.

It is specifically contemplated that the composition can be configured as a patch to be applied to the skin. In this case, the α_{1C} modulator can be administered through the skin over a period of time up to 6 weeks.

In certain embodiments, a composition is formulated with about, at least about, or at most about 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 ng of an α_{1C} modulator, or any range derivable therein.

Alternatively, the composition may be formulated as a suppository, such as a rectal suppository.

Whether formulated for time release or sustained release, the amount of the α_{1C} modulator administered will be about or less than about 25 nM/kg/day during or throughout the time period. Other amounts discussed above are contemplated as well.

The present invention also concerns methods for screening for a candidate therapeutic agent for a gastrointestinal motility disorder. In some embodiments, the method comprises: a) contacting a cell with the candidate therapeutic agent; b) assaying for gene expression of the α_{1C} subunit of the L-type calcium channel in the cell; and, c) comparing the levels of gene expression of the α_{1C} subunit in the presence and absence of the candidate therapeutic agent. In some cases, a control assay in the absence of the candidate therapeutic agent is conducted at the same time the candidate agent is assayed. It is contemplated that the candidate therapeutic agent may be a protein, nucleic acid, or small molecule. Moreover, it is contemplated that screening assays may be employed with a library or array.

Additionally, screening methods may further include administering the candidate therapeutic agent to an animal whether to test it as a candidate substance, to evaluate it for therapeutic efficacy or toxicity/safety, for quality control, or for a therapeutic benefit.

Other steps or methods of the invention include manufacturing the candidate therapeutic agent.

An “effective amount” of the pharmaceutical composition, generally, is defined as that amount sufficient to detectably and repeatedly to achieve the stated desired result, for example, to increase or decrease gene expression of α_{1C} subunit at least 10% for a period of at least about 2 hours or to ameliorate, reduce, minimize or limit the extent of a gastrointestinal disease or disorder, or its symptoms. More rigorous definitions may apply, including elimination, eradication or cure of a disease or disorder.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Show Q-PCR assessment of α_{1C} mRNA expression relative to exposure to potential neurotransmitters of enteric motor neurons. (A) VIP, (B) PACAP, (C) 8-bromo cAMP, (D) ACh, SP, ATP and s-nitrosoglutathione.

FIGS. 2A-2C. Shows cAMP levels in HCCSMC for 24 h after treatment with forskolin, PACAP, or VIP.

FIGS. 3A-3D. Show the kinetics of (A) VIP-induced expression of α_{1C} mRNA, (B) Isoproterenol-induced expression of a mRNA, (C) VIP-induced α_{1C} protein expression, and (D) α_{1C} protein expression relative to VIP concentration.

FIG. 4. VIP enhanced the mRNA of α_{1C}. H-89 had no effect on its own, but it partially inhibited the effect of VIP.

FIG. 5. VIP-treatment for 24 h enhanced Ca²⁺ influx in HCCSMC by 60 μmM KCl measured by fura-2 AM.

FIG. 6. VIP-treatment of human colonic circular muscle strips for 24 h increased their contractile response to ACh.

FIG. 7. Systemic administration of VIP receptor antagonist (p-chloro-D-Phe⁶,Leu17)-VIP by osmotic pump for 3 days suppressed the contractile response of rat middle colon muscle strips to ACh. n=3

FIG. 8. VIP concentration-dependently increased α1C1b promoter reporter activity in HCCSMC. n=3

FIGS. 9A-9B. Transient transfection with siRNA of CREB decreased abundance of CREB (n=2). VIP enhanced promoter activity in normal cells, but this enhancement was blunted in cells transfected with CREB siRNA (n=2). (A) RNA levels and (B) normalized luciferase activity.

FIG. 10. Pull-down assay indicated that CREB binds to both CRE1 and CRE2. TNFα reduces the binding after VIP treatment.

FIG. 11. Deletion analysis assessed by activity of a luciferase reporter and assessment of reporter activity in response to VIP. (A) CRE elements shaded and (B) κB elements shaded.

FIG. 12. ERK ½ and p38 antagonists had little effect on enhancement of promoter activity by VIP. On the contrary, the JNK antagonist father enhanced it. The JNK antagonist also enhanced promoter activity by itself.

FIG. 13. VIP enhanced promoter activity, while TNFα suppressed it. VIP partially blocked the suppression of promoter activity by TNFα. n=3

FIG. 14. TNFα suppressed the contractile response to ACh, whereas, VIP enhanced it. VIP also partially reversed the suppression of contractile response to TNFα. n=3

FIG. 15. Sub-threshold dose of TNBS, 30 mg/kg, suppressed the contractile response to ACh much less than the fill dose of 130 mg/kg. VIP receptor antagonist administered by an osmotic pump by itself suppressed the contractile response and given with the sub threshold dose of TNBS, it increased the suppression of contractile response greater than that with the full dose of TNBS. n=3.

FIG. 16. Shows an exemplary immunoflourescence study of phosphorylated CREB (pCREB). DIC—differential interference contrast microscopy, DAPI—DNA specific fluorescent stain, Alexa 488—fluorophore conjugated to pCREB specific antibody.

FIG. 17. Shows VIP induced phosphorylation of CREB with (A and B) no inhibitor, PKA inhibitor—H89, and protein kinase C inhibitor calphostin C; (C) VIP, PKA inhibitor—H89, adenylate cyclase agonist—forskolin, and p200; and (D) time course of VIP treatment.

FIG. 18. Shows NFκB translocation and binding in the presence of VIP, VIP+TNFα at 0, 1, 6, or 24 hours.

### DESCRIPTION OF ILLUSTRATIVE EMBODIMENTS

The present invention concerns methods and compositions for altering the expression of α_{1C} subunit of L-type calcium channels in smooth muscle cells, particularly gastrointestinal smooth muscle cells. Such methods and compositions can be readily employed for diseases and conditions that are caused or affected by gastrointestinal motility.

### 1 L-Type Calcium Channels

The L-type Ca²⁺ channels (Caᵥ1.2) are expressed ubiquitously in excitable cells. By regulating influx of Ca²⁺, they mediate numerous cellular processes and functions, including excitation-contraction coupling in muscle cells, excitation-secretion coupling in epithelial and immune cells, neurotransmitter release in neurons and cell proliferation and gene expression in most cell types. These channels were first cloned from rabbit skeletal muscle (Tanabe et al., 1987). They are pentameric complexes composed of α1, β, γ, and disulfide-linked α2/δ subunits (Catterwall, 1988; Ellis et al., 1988; Ruth et al., 1989; Jay et al., 1990). The 190-250 kDa α1 is the autonomous pore-forming subunit that contains the voltage sensing and gating mechanisms as well as binding sites for dihydropyridines (Perez-Reyes et al., 1989; Kim et al., 1992; Murthy et al., 1996). The remaining subunits of the channel modulate the characteristics and expression of the α1 subunit (Abernethy and Soldatov, 2002; Mikami et al., 1989). Four types of L-type calcium channels encoded by different genes have been cloned: Caᵥ1.1 (α1S) expressed in skeletal muscle cells, Caᵥ1.2 (α1C) expressed in heart, vascular and visceral smooth muscle cells, Caᵥ1.3 (α1D) expressed in neuroendocrine tissue and Caᵥ1.4 (α1F) expressed in the retina.

Further molecular diversity of Caᵥ1.2 channels is achieved by alternative gene splicing and regulation of transcription by alternative promoters in the 5′ flanking regions of exon 1a, exon 1b and exon 2 (Soldatov, 1992; Pang et al., 2003; Saada et al., 2003). The expression of Caᵥ1.2 channels in human cardiac myocytes is regulated predominantly by transcripts containing exon 1a and its 5′ flanking region (promoter α1C1a) and a smaller proportion of transcripts containing exon 1b and its 5′ flanking region (promoter α1C1b). (Dai et al., 2002; Mikami et al., 1989). It was reported recently that the expression of Caᵥ1.2 channels in HCCSMC is regulated almost entirely by the promoter α1C1b (Saada et al., 2003).

Several studies have reported that the expression of L-type Ca²⁺ channels and calcium currents are suppressed during inflammation, resulting in reduced Ca²⁺ influx (Liu et al., 2001; Akbarali et al., 2000). The reduction of Ca²⁺ influx leads to suppression of rhythmic phasic contractions and tone that is, in part, responsible for the symptom of diarrhea in colonic inflammation (Sarna, 1991; Sethi and Sarna, 1991). The reduction of Ca²⁺ influx is due to decrease in the expression of the pore-forming α_{1C} subunit of L-type Ca²⁺ channels. More recent studies (Liu et al., 2001) show that the suppression of α_{1C} in inflammation and in response to TNFα, a potent inflammatory response mediator, is due to the activation of transcription factor NF-κB, which is a critical transcription factor in inflammatory response. See also Examples. Numerous studies have shown that in inflammation, NF-κB induces the expression of inducible inflammatory mediators, including cytokines, chemokines, growth factors, inducible nitric oxide synthase, cyclooxygenase and cell adhesion molecules (Siebenlist et al., 1994; Barnes and Karin, 1997; Neurath et al., 1998; Ghosh et al., 1998). The p65 NF-κB subunit plays a prominent role in the transcription of the above genes because of its transactivation domain (Ghosh et al., 1998). This domain is absent in the p50 subunit, which makes it largely a repressor of its target genes (Ghosh et al., 1998).

The studies found, however, that for a constitutively expressed protein, such as α_{1C} subunit of L-type Ca²⁺ channels, both p65 and p50 subunits of NF-κB are repressor subunits (see Examples). Upon their translocation to the nucleus in response to TNFα, they bind to two variant κB motifs at −1243/−1234 and −1225/−1216 in the α1C1b promoter and repress its constitutive expression. It was also found by using promoter reporter assays that concurrent binding of p50/p65 to both the κB motifs was essential for the repression of α_{1C}. The repressive role of the above two κB binding sites on α1C1b promoter was confirmed by progressive 5′ deletions of the promoter and point mutations. It was established also that the repression of α_{1C} protein by TNFα leads to suppression of contractility in response to ACh in a muscle bath environment. Transfection of the muscle strips by p50 and p65 antisense oligonucleotides prior to their 24 h treatment with TNFα reversed the suppression of contractility in circular smooth muscle strips (See Examples).

α_{1C} is the pore-forming subunit of L-type calcium channels. The influx of Ca²⁺ through these channels is an immediate early step in cell signaling that regulates excitation-contraction coupling. The regulation of the expression of these channels is, therefore, of critical importance in maintaining normal Ca²⁺ influx and to modulate it in response to inflammatory and environmental stressors (Liu et al., 2001). In addition, there is natural degradation of cellular proteins with time and by endogenous proteases. The genes encoding these proteins therefore, need to be expressed continually, periodically or on a need basis to restore them to normal levels. Several such transcriptional processes are regulated by external stimuli.

In a recent study, Abad et al. (Abad et al., 2003) reported that systemic administration of VIP bolus injections exhibits therapeutic effects on TNBS (trinitrobenzene sulfonic, acid)-induced colitis in a mouse model. Particularly, they reported that VIP abrogated TNBS-induced diarrhea and it reduced macroscopic and microscopic inflammation. They did not investigate whether the abrogation of diarrhea was associated with prevention of colonic motility dysfunction or whether it was secondary to partial abrogation of the inflammatory response nor did they discuss whether the expression of the α_{1C} subunit was affected in smooth muscle cells. It is noteworthy that VIP in this study did not completely suppress MPO (myeloperoxidase) activity.

It is noteworthy that in experimental models of inflammation, the VIP content of the myenteric plexus is diminished in the early stages of inflammation, which may allow the myogenic inflammation to proceed (Miampamba and Sharkey, 1998). The VIP content begins to increase in 5-7 days, which may help restore the expression of α_{1C} subunit of L-type Ca²⁺ channels and hence normalize motility function.

### II Smooth Muscle Cell Disorders

The present invention concerns methods and compositions that are relevant to the contractility of smooth muscle cells. Smooth muscle cells can be found throughout the body, including along the gastrointestinal tract, in the lining of the vasculature, and around organs.

A Gastrointestinal Motility Disorders

The science of gastrointestinal motility has made phenomenal advances during the last fifty years. Yet, there is a paucity of effective promotility drugs to treat functional bowel disorders that affect 10% to 20% of the U.S. population. A part of the reason for the lack of effective drugs is our limited understanding of the etiology of these diseases. In the absence of this information, mostly an ad hoc approach has been used to develop the currently available drugs, which are modestly effective or effective in only a subset of the patients with functional bowel disorders.

The development of the next generation of promotility drugs is based on our current understanding of: 1) the different types of contractions that produce overall motility function of mixing and orderly net distal propulsion in major gut organs; 2) the regulatory mechanisms of these contractions; 3) which receptors and intracellular signaling molecules could be targeted to stimulate specific types of contractions to accelerate transit and; 4) the strengths and limitations of animal models and experimental approaches that could screen potential promotility drugs for their efficacy in human gut propulsion in functional bowel disorders.

Most currently available promotility drugs or those under development seem to have their origins in a known physiological or pharmacologic effect or in their established role in a different system. For example, CCK receptor antagonists (loxiglumide and deloxyglumide) are thought to be potential promotility drugs because fat-induced CCK release delays gastric emptying. Therefore, the thinking is that blockade of this delay would enhance the rate of gastric emptying (Schwizer et al., 1997). Similarly, peripherally acting 1-opioid receptor antagonists have been proposed as promotility drugs to reduce the duration of post-operative ileus, but post-operative ileus is not only due to the use of opioids as analgesics; the overdrive of sympathetic system plays a prominent role. These drugs would have worked fine in functional bowel disorders if delayed transit in these patients were due to excess production of endogenous CCK or opioids, but that is not the case. More importantly, these antagonists do not actively stimulate gut contractions whose impairment may be the root cause of delayed transit in functional bowel disorders.

5-HT₄ receptor agonists were tested in the eighties based largely on the hunch that 5-HT plays a prominent role in the CNS. Although the CNS and the enteric nervous system (ENS) share several common neurotransmitters, their respective functions and networking have more dissimilarities than similarities. Further testing of 5-HT₄ agonists indicated that they stimulate the classic peristaltic reflex, i.e. ascending excitation and descending inhibition in in vitro experiments in rodents and guinea pigs (Grider et al., 1996). This observation was taken as supportive evidence of the suitability of these agonists as promotility drugs. However, in non-rodent and non-guinea pig species, especially in humans, most postprandial propulsion of digesta does not occur by the classic peristaltic reflex, i.e. ascending contraction and descending inhibition; instead it occurs by rhythmic phasic contractions (RPCs) that do not produce descending inhibition (Cowles and Sarna, 1990; Sarna et al., 1989). This ad hoc approach may be one of the reasons that the currently available 5-HT₄ agonists are modestly effective or effective only in a subset of patients with functional bowel disorders in which transit is delayed in specific organs. A complete understanding of the mechanisms by which existing promotility drugs work is also lacking. The understanding of these mechanisms can be helpful in modifying these drugs or in testing of alternate drugs to obtain greater efficacy in a broader population of functional bowel disorder patients.

The circular smooth muscle cells in intact animals generate three distinct types of contractions for distal propulsion of digesta (Sarna and Shi, 2006): 1) Rhythmic phasic contractions (RPCs); 2) Giant migrating contractions (GMCs), and; 3) Tonic contractions (TCs). The function, regulation, and spatial and temporal characteristics of these contractions differ.

Rhythmic Phasic Contractions (RPCs): These contractions mix the ingested meal with exocrine, endocrine and mucosal secretions, and propel the digesta distally at relatively slow rates so that adequate time is available for digestion and absorption of nutrients. The RPCs are absent in the normal esophagus because this organ does not require mixing, digestion or absorption. The three important characteristics of these contractions that determine their propulsive efficacy are: a) mean propagation distance in the distal direction; b) mean amplitude and; c) mean frequency of contractions (Cowles and Sarna, 1990). The distal propagation of RPCs is the most critical parameter that determines postprandial propulsion. Non-propagating (randomly occurring) RPCs or RPCs that propagate over very short distances cause mostly back and forth movements of digesta. The non-propagating contractions frequently turn over the luminal contents, mix them with secretions and expose the mixture uniformly to the absorptive mucosal surface. The propagation distance of RPCs depends on two factors: 1) the distance over which the slow waves are phase-locked and; 2) the contiguous distance over which the cholinergic motor neurons concurrently release acetylcholine (ACh) (Sarna, 1989). The mean propagation distance of propagation of RPCs decreases steadily from the stomach to the rectum, which manifests as slower propulsion rates distally in the gut. This organization is consistent with the digestive and absorptive functions of the stomach, small intestine and colon. The maximum mean distance of propagation of RPCs in the stomach and the duodenum, where the slow raves are generally phase-locked, is still only a few cm. The RPCs propagate very little in the colon.

A larger amplitude of RPCs enhances their efficacy of propulsion by a greater or complete occlusion of the lumen so that the digesta does not escape through the partial opening of the lumen and left behind during propulsion by a propagating RPC. The frequency of propagating RPCs determines how many times digesta is propelled per unit time and hence the total volume of propulsion in a given time period.

Giant Migrating Contractions: GMCs are large-amplitude and long-duration ultra-propulsive contractions that strongly occlude the lumen and rapidly propagate over long distances in the esophagus, small intestine and the colon (Chey et al., 2005; Karaus ans Sarna, 1987; Matsushima, 1989; Sarna, 1987). The GMCs do not occur in the stomach. In the esophagus, these contractions occur after each swallow; they also occur spontaneously in the distal esophagus to rapidly clear the refluxed acid. In the small intestine and colon, these contractions occur spontaneously in the terminal ileum and the proximal colon about 2 to 5 times a day (Annese et al., 1997; Bamplton et al., 2000; Bassotti et al., 2004; Clemens et al., 2003; Karaus and Sarna, 1987; Otterson and Sarna, 1994; Rao et al., 2001; Sarna, 1987). These contractions also precede defecation and provide the force for rapid evacuation of feces. The large lumen occluding amplitude of GMCs, their long duration and rapid propagation over long distances produce mass movements of the digesta.

The strong compression of the gut wall by the large amplitude of GMCs activates the sensory receptors to trigger descending inhibition of spontaneously occurring RPCs and relaxation of muscle tone (Bassotti et al., 1999; Chey et al., 2001; Otterson and Sarna, 1994). This descending inhibition facilitates mass movements in two ways. The inhibition of spontaneous RPCs in the segment distal to a GMC reduces the resistance to rapid propulsion of digesta by the GMC. Furthermore, relaxation of the distal segment allows it to distend without an increase in tone to accommodate the large volume of digesta being propelled rapidly and without triggering nociceptors. The descending inhibition triggered by GMCs in respective organs also relaxes the lower esophageal sphincter (LES), ieo-cecal junction and internal anal sphincter to let the luminal contents pass through without resistance (Bassotti et al., 1999; Matsufuji and Yokoyama, 2003). The spontaneously occurring RPCs in the sigmoid colon/rectum and ileum do not produce descending inhibition to relax the internal anal and ileo-cecal sphincters respectively. Note also that the descending relaxation of the LES is impaired when GMCs are replaced by phasic contractions in achalasia and diffuse esophageal spasm. A defect in the inhibitory innervation of the LES may also contribute to impaired relaxation of the LES in these motility disorders.

Tonic Contractions (TCs): The increase of tone that decreases the luminal diameter, by itself has little or no effect on mixing or propulsion in the small intestine and the colon. However, as a result of decrease in luminal diameter by increase in tone, the same amplitude of RPCs would occlude the lumen more effectively and therefore be more effective in propulsion. The tone of the small intestine and colon is increased after ingestion of a meal (Coffin et al., 1994; Ford et al., 1995; Jouet et al., 1998). The cellular signaling pathways for excitation-contraction coupling to generate tone are different from those that generate RPCs and GMCs (Huang et al., 2005; Sarna, 2000).

In particular embodiments, the invention relates to the contractility and relaxation of gut smooth muscle cells. Gut smooth muscle cells include smooth muscle along the gastrointestinal tract, as well as those in the mouth. To that extent, the present invention concerns diagnostic, preventative and therapeutic applications for gastrointestinal motility disorders, specifically including functional bowel disorders for which no organic cause or etiology has yet been identified, but that is ultimately a result from a gastrointestinal motility disorder.

Gastrointestinal motility disorders are characterized by irregular or abnormal contraction of the muscles that mix and propel the contents of the gastrointestinal tract. Such disorders include, but are not limited to, irritable bowel syndrome (IBS), inflammatory bowel disease, constipation, diarrhea, gastroparesis—both diabetic and idiopathic, abdominal pain, abdominal bloating, intestinal dysmotility, chronic intestinal pseudo-obstruction (CIP), small bowel bacteria overgrowth, pelvic floor dyssynergia, fecal incontinence, Hirschsprung's disease, achalasia, scleroderma, dysphagea, Chagas disease, gastroesophageal reflux disease (heartburn), and symptomatic diffuse esophageal spasm.

It will be understood that the present invention particular includes the treatment of those gastrointestinal motility disorders in which induction of relaxation or contraction of gut smooth muscle cells will alleviate, reduce, eliminate, or prevent symptoms of the disorder or the disorder itself.

1 Strategies for the Design and Development of Gut Promotility Drugs:

Gut transit can be accelerated by enhancing the amplitude, frequency and mean propagation distance of RPCs; by stimulating GMCs; and by increasing smooth muscle tone. The two basic steps in the development of an effective promotility drug are: 1) Identify the type(s) of contractions that should be stimulated to accelerate transit in the desired gut organ; 2) Identify the most suitable receptor and/or signaling molecule whose activation would preferably stimulate that contraction(s).

2 Suitability of Specific Types of Gut Contractions to Accelerate Transit

RPCs: The postprandial transit of digesta may be delayed due to a decline in the overall incidence of RPCs resulting in a decrease in the mean amplitude and frequency of contractions. For this condition, the RPCs can be an effective target for stimulation by promotility drugs to restore normal transit. However, in several functional bowel disorders, the amplitude and/or frequency of postprandial RPCs may not be decreased or they may even be enhanced (Bueno et al., 1980). The delay in transit in these conditions is due to a decrease in the mean distance of propagation of RPCs or near total absence of propagating RPCs (Cook et al., 2000). The stimulation of RPCs in these conditions may be ineffective in accelerating transit or it may further retard transit due to the stimulation of non-propagating RPCs. The propagation distance of RPCs depends upon the distance over which slow waves are phase-locked. Due to poor electrical coupling among circular muscle cells in the terminal ileum and colon, the slow waves are largely phase-unlocked. Therefore, the stimulation of RPCs for accelerating transit in these parts of the gut may be counter productive or marginally effective.

The stimulation of RPCs is an effective target for the acceleration of gastric emptying in conditions, such as diabetic or idiopathic gastroparesis. However the regulation of gastric emptying is multifactorial and complex. It depends on coordination among several mechanisms, including pyloric contractions and tone, antro-pyloro-duodenal coordination, fundic adaptive relaxation followed by gradual increase in tone, and propagating RPCs in the body of the stomach (Haba and Sarna, 1993; Orihata and Sarna, 1994). It is, therefore, critical that the stimulation of gastric RPCs by promotility drugs does not adversely affect the other regulatory mechanisms so as to negate their beneficial effects (Sarna et al., 1991). For example, concurrent stimulation of RPCs in the duodenum or increase of pyloric tone and RPCs may deteriorate antro-pyloro-duodenal co-ordination and adversely affect the rate of gastric emptying (Haba and Sarna, 1993; Sarna et al., 1991). In this regard, erythromycin that accelerates the rate of gastric emptying stimulates the postprandial RPCs in the stomach but it suppresses them in the duodenum (Sarna et al., 1991).

The stimulation of RPCs can also be effective in restoring the normal rate of transit in small intestine and colon when RPCs are suppressed in conditions, such as idiopathic intestinal pseudoobstruction or megacolon.

GMCs: The stimulation of GMCs is an ideal target to accelerate colonic transit in functional bowel disorders, such as idiopathic constipation and constipation predominant irritable bowel syndrome (IBS-C). The advantages of stimulating colonic GMCs in these conditions are: 1) they are very effective in mass propulsion over long distances because their generation and propagation do not depend upon slow waves. Therefore, any defect in the generation or phaselocking of slow waves would not affect the rapid propulsion by GMCs. 2) The GMCs in the distal colon produce descending inhibition to relax the internal anal sphincter. This would facilitate defecation and partially or completely overcome outlet obstruction (Bassotti et al. 1999). The stimulation of RPCs would not achieve this effect. 3) The strong propulsive force of a GMC can propel hardened or impacted feces due to constipation; the stimulation of RPCs may not achieve this effect.

The limitations of stimulating GMCs to accelerate transit are: 1) over stimulation of GMCs could produce frequent mass movements and hence diarrhea. This limitation may be overcome by adjusting the dose of the promotility drug. 2) In patients with visceral hypersensitivity, the stimulation of GMCs may exacerbate the sensation of abdominal cramping. This may happen if the descending inhibition is defective (Sarna and Shi, 2006).

The ultra-rapid propulsion of bolus in the esophagus is produced by a GMC that follows a voluntary swallow. In conditions such as achalasia and diffuse esophageal spasm, the GMCs are replaced by simultaneous or randomly occurring smaller amplitude RPCs that are ineffective in rapid propulsion of the swallowed bolus. In addition, the absence of spontaneous secondary GMCs in the distal esophagus in response to acid reflux may impair its rapid and effective clearance and contribute to the development of esophagitis. The stimulation of esophageal GMCs, therefore, would be an attractive target for relieving the symptoms of gastroesophageal reflux, achalasia and diffuse esophageal spasm.

The stimulation of GMCs in the small intestine would also accelerate transit. However, most absorption of nutrients occurs in this organ and rapid mass movements produced by GMCs would deprive the digesta of adequate time required for digestion and absorption. The ultra-rapid emptying of nutrients from the small intestine into the colon may also increase osmotic load and result in diarrhea. Therefore, stimulation of GMCs to accelerate postprandial transit in the small intestine may not be a desirable target, except on a short term basis. The GMCs do not occur in gastric smooth muscle cells and, therefore, they cannot be the targets of promotility drugs to accelerate gastric emptying.

Tonic Contraction: The increase of tone only indirectly enhances the transit rate in the small intestine and the colon by enhancing the efficacy of RPCs. However, the increase of postprandial fundic tone is an effective target to enhance the rate of gastric emptying. The increase of postprandial fundic tone (or the blockade of adaptive relaxation) would transfer the fresh digesta more rapidly from the fundus to the body of the stomach in preparation of its emptying by gastric RPCs. However, an increase in postprandial tone may reduce food intake because of early satiety signals.

3 Enteric Neurons and/or and Circular Smooth Muscle Cell as Targets of Promotility Drugs:

The regulatory mechanisms of neurotransmitter release from the enteric neurons and excitation-contraction coupling in circular smooth muscle cells together determine the types of gut contractions generated and their spatio-temporal characteristics. Therefore, they serve as the most suitable targets of promotility drugs.

The end point of enteric neural regulation of motility function is the release of acetylcholine (ACh) by cholinergic excitatory motor neurons, and release of nitric oxide (NO) and vasoactive intestinal polypeptide (VIP) by the non-adrenergic non-cholinergic (NANC) inhibitory motor neurons. The motor neurons (S type) receive inputs from interneurons, intrinsic sensory neurons (ISNs) and intrinsic spontaneously active neurons (ISANs) at nicotinic receptors (Sarna and Shi, 2006). The ISANs spontaneously generate excitatory postsynaptic potentials (EPSPs).

The cell bodies of these neurons are localized in the myenteric plexus and they innervate the motor neurons directly or through interneurons. On the other hand, the ISNs whose cell bodies are in the myenteric and submucosal plexi and sensory endings in the mucosal layer generate EPSPs largely in response to stimulation, such as that produced by nutrients in the lumen or mechanical stimulation of the mucosa. The Phase II and Phase III contractions of the migrating motor complex in the fasting state result from spontaneous activity of ISANs (Lomax et al., 1999; Morse and Sassone-Corsi, 2002). The trigger for the release of neurotransmitters to stimulate Phase II and Phase III contractions in the interdigestive state does not come from the ISNs. There is no nutritional digesta in the gastric and small intestinal lumen during the interdigestive state.

ACh acts mainly on muscarinic M3 receptors on circular smooth muscle cells (Shi and Sarna, 1997), VIP on VPAC2 receptors (Murhty and Maklouf, 1994), and NO permeates through the membrane to activate soluble guanylyl cylase. The M3 and VPAC2 are G protein coupled receptors through which they activate multiple intracellular signaling pathways (Makhlouf and Murthy, 1997; Sarna and Shi, 2006). The signaling pathways activated by these neurotransmitters are determined by their respective receptor subtypes and associated G proteins, as well as by the amount and duration of accumulation of the neurotransmitter at the neuroeffector junction (Biancani et al., 1994; Sarna, 2003). NO activates its signaling pathways through cGMP. The end point of activation of intracellular signaling pathways is phosphorylation of 20 kD myosin light chain (MLC₂₀), which initiates cross-bridge cycling and smooth muscle contraction. The intensity and duration of MLC₂₀ phosphorylation determine the amplitude and duration of contraction. In a simplistic way MLC₂₀ is phosphorylated by myosin light chain kinase (MLCK) and dephosphorylated by myosin light chain phosphatase (MLCP). The net intensity and duration of phosphorylation of MLC₂₀, therefore, depends on the relative intensities and time courses of MLCK and MLCP phosphorylations. The signaling pathways stimulated by ACh phosphorylate MLCK and dephosphorylate MLCP concurrently to enhance the phosphorylation of MLC₂₀ (Somlyo and Somlyo, 2003 and 2000). The signaling pathways stimulated by NO and VIP dephosphorylate MLCP to enhance its activity as well as to decrease [Ca²⁺]i and MLCK phosphorylation. Therefore, the type of contraction stimulated by a promotility drug is determined by the kinetics of excitatory and inhibitory neurotransmitter release by enteric motor neurons and by the activation of intracellular signaling pathways that compete for the phosphorylation of MLC₂₀ in circular smooth muscle cells. The strategies that may help in selecting enteric neuronal receptors and intracellular signaling molecules in circular smooth muscle cells to stimulate the three types of contractions.

4 Molecular and Pharmacological Targets of Promotility Drugs

Slow transit may occur due to a defect in the release of excitatory and inhibitory neurotransmitters from the enteric motor neurons and/or due to a defect in the excitation/contraction coupling in circular smooth muscle cells. The following strategies can be used in the development of an effective promotility drug (such as VIP, PACAP, or norepinephrine) to accelerate transit using both of these cell types as targets:

In normal gut motor function, the motor neurons receive inputs directly from ISNs, ISANs or indirectly through interneurons to release excitatory and inhibitory neurotransmitters. Together, these neurons are referred to as presynaptic neurons. Numerous types of receptors have been identified on presynaptic neurons by using in vitro pharmacological and electrophysiological approaches (Bornstein et al, 2004; Galligan, 2002; Wood, 1994). However, the specific receptor types or subtypes that mediate the in vivo postprandial release of ACh remain unknown due to the limitations of in vivo measurements. Nevertheless, all those receptor types on presynaptic neurons that release ACh from the excitatory motor neurons in vitro are potential candidates for the stimulation of RPCs, GMCs and increase of tone. The current knowledge of the site of neuronal defects that retard gut transit in motility disorders, such as gastroparesis, idiopathic constipation and constipation-predominant IBS, is severely limited. In the absence of this knowledge, it may be prudent to pick a neuronal receptor target that is located as close as possible to the motor neurons. If the neuronal defect is between the receptor to be stimulated and the motor neurons, the defect is likely to impair the efficacy of the promotility drug in patients, even though it demonstrates efficacy in normal healthy subjects. In this regard, 5-HT₄ agonists that are thought to act on 5-HT₄ receptors on mucosal sensory nerve endings of ISANs may be at a disadvantage. These receptors are located farthest away from the motor neurons. This may be one of the reasons that 5-HT₄ agonists are effective only in a subset of IBS-C patients.

Although gut smooth muscle cells contract upon the release of ACh from the excitatory cholinergic motor neurons, exogenous cholinergic agonists are ineffective in accelerating transit by the stimulation of R₅-PCs. These drugs bypass the enteric nervous system and they act concurrently and directly on circular muscle cells everywhere, resulting in stimulation of simultaneous or non-propagating RPCs. Furthermore strong stimulation of muscarine receptors on smooth muscle cells may uncouple slow waves at adjacent sites and suppress propagation of contractions that would further retard transit. Cholinesterase inhibitors that accumulate Ach also stimulate non-propagating RPCs for the same reason. However, as noted above, the accumulation of ACh at the neuroeffector junction may also stimulate GMCs, which are highly propulsive irrespective of slow waves (Frantzides et al., 1987; Karaus and Sarna, 1987).

5 Specific Strategies for Stimulation of RPCs

The postprandial RPCs that propel digesta occur intermittently as single contractions or as groups of a few contractions of variable amplitude (Cowles and Sarna, 1990; Johnson et al., 1997; Lomax et al., 1999). This spatial pattern of contractions is most effective in producing mixing and slow net distal propulsion. The intermittent occurrence of contractions of variable amplitude may be due to either the intermittent release of ACh or variable release of the competing excitatory and inhibitory neurotransmitters ACh and NO/VIP. The latter probability is consistent with the data that the interneurons, ISNs and ISANs provide inputs to both the excitatory and inhibitory motor neurons at nicotinic receptors. Accordingly, a preferred target of promotility drugs would be those presynaptic neurons that innervate both the excitatory and inhibitory motor neurons. The simulation of RPCs at their maximal rate by targeting only the excitatory motor neurons may be counter productive.

Concurrent recordings of excitatory postsynaptic potentials (EPSPs) from the enteric neurons and the contractions that they stimulate are not available. However, it seems likely that RPCs, whose duration is short when compared with those of GMCs or TCs, are stimulated by a single or a group of fast EPSPs. The S type interneurons, but not the S type motor neurons are therefore the attractive targets of promotility drugs to stimulate intermittently propagating RPCs of variable amplitude. The presynaptic locus of action of a potential promotility drug, therefore, must be ascertained.

The intermittent spatiotemporal patterns of Phase II RPCs are nearly as effective in propulsion as the postprandial contractions (Sarna et al., 1989; Summers et al., 1976). Therefore, a drug that stimulates Phase II-like or Phase III-like contractions in the interdigestive state likely acts on presynaptic ISANs to stimulate RPCs and is, therefore, likely to accelerate postprandial transit in the stomach and small intestine. Note that Phase III-like contractions do not occur in the postprandial state. The stimulation of Phase III-like contractions by a potential promotility drug is only an indication of its locus of action on presynaptic ISANs. In this regard, motilin and erythromycin that stimulate

Phase II-like and Phase III-like contractions in the interdigestive state have demonstrated potential in accelerating postprandial transit in the stomach and the small intestine. In the postprandial state, the phase II-like contractions triggered by ISANs are potentiated by additional input to the motor neurons due to the activation of ISNs by digesta.

The RPCs result from competing inputs to the smooth muscle cells for excitation/contraction coupling by the excitatory (ACh) and inhibitory neurotransmitters (NO/VIP). Theoretically, the RPCs can be enhanced either by the release of ACh or inhibition of NO and VIP. However, most studies show that inhibition of the inhibitory nitrergic neurons retards gastric emptying although it stimulates the RPCs (Orihata and Sarna, 1994). This is due to the selective stimulation of pyloric and duodenal contractions by unopposed action of ACh resulting in the impairment of antro-pyloro-duodenal coordination (Orihata and Sarna, 1994 and 1996). The blockade of inhibitory neurons, thus, may not be an effective target of promotility drugs.

Slow waves determine the timing, maximum frequency and maximum propagation distance of RPCs. Electrical stimulation of slow waves by implanted electrodes has been reported to accelerate the rate of gastric emptying in normal animals (Chen et al., 2005). However, this method may not work if the delayed gastric emptying is due to the impaired release of ACh or due to a defect in excitation/contraction coupling. Slow waves, by themselves, do not generate contractions. It requires the release of ACh during membrane depolarization to generate an RPC (Sarna, 1989). Furthermore, electrical stimulation can increase slow wave frequency only marginally and when the slow wave frequency is increased the distance over which they are phase-locked decreases in the small intestine (Sarna and Daniel, 1975 and 1973), resulting in reduced distance of propagation of RPCs.

On the contrary, reversing the direction of propagation of slow waves in the stomach or the duodenum or by stimulating them in pyloric smooth muscle cells to disrupt antro-pyloroduodenal coordination can effectively retard the rate of gastric emptying (Liu et al., 2005; Yao et al., 2005). This may have beneficial effects by inducing early satiety in obese patients (Yin and Chen, 2005).

Concurrent electrical stimulation of enteric neurons and slow waves has been largely unsuccessful in accelerating intestinal transit because ACh release by electrical stimulation of neurons is not similar to its spontaneous intermittent release under physiological conditions. Therefore, the spatial pattern of RPCs produced by concurrent electrical stimulation of slow waves and enteric neurons does not mimic the intermittent spatio-temporal pattern of postprandial contractions. Furthermore, electrical stimulation of enteric neurons can be accomplished only at a few discrete locations, not over the entire organ.

6 Specific Strategies for Stimulation of GMCs

Most evidence indicates that excessive release or accumulation of ACh at the neuroeffector junction stimulates GMCs (Frantzides et al., 1987; Karaus and Sarna, 1987; Sarna, 2000). The excessive release of ACh may be due to the stimulation of slow EPSPs that can generate a long series of action potentials lasting longer than 10 seconds (Palmer et al., 1986; Wood, 1994). Close intra-arterial administration of CGRP that stimulates sEPSPs consistently generates GMCs in the small intestine (Sarna, 2000). Systemic administration of guanethidine also transiently generates GMCs that is likely due to blockade of the inhibitory effect of norepinephrine on the release of ACh at the presynaptic terminals (Tsukamoto et al., 1997). Likewise, systemic or close intra-arterial administration of anticholinesterase neostigmine stimulates GMCs (Frantzides et al., 1987; Karaus and Sarna, 1987). In all these cases, the GMCs propagate distally and produce mass movements. The receptors that stimulate GMCs are not the same in the small intestine and the colon. For example, CGRP stimulates GMCs in the small intestine, but not in the colon (Sarna, 2000; Tsukanoto et al., 1997). On the other hand, close intra-arterial administration of substance P stimulates GMCs in the colon, but not in the small intestine (Jouet and Sarna, 1996). Substance P, however, acts directly on smooth muscle cells to stimulate colonic GMCs (Tsukamoto et al., 1997). As noted earlier, the propagation of GMCs does not depend upon slow waves. Therefore, either smooth muscle or enteric neural receptors are effective targets to stimulate them to enhance transit. In the neurons, the compounds that stimulate slow EPSPs may be more effective in generating GMCs. The stimulation of GMCs by a direct action of promotility drugs on circular smooth muscle cells is an attractive option in cases where the delayed transit is due to neural defects. Furthermore, a GMC can enhance transit regardless of whether the delayed transit is due to the suppression of RPCs or enhancement of nonpropagating RPCs.

Accumulating evidence over the last two decades shows that the signaling pathways for excitation/contraction coupling in smooth muscle cells differ for the generation of the three types of gut contractions (Huang et al., 2005; Sarna, 2000). This opens up tremendous opportunities for targeted activation of intracellular signaling molecules by promotility drugs to selectively stimulate a specific type or types of contractions, particularly the GMCs that do not depend upon slow waves for their propagation. As noted above, the targeting of intracellular signaling molecules for excitation/contraction coupling is particularly attractive if slower transit is due to impaired neurotransmitter synthesis or release from the motor neurons. In this case, pharmacologic stimulation of receptor on presynaptic neurons may yield limited beneficial effect in patients.

Recent studies show that the expression of receptors, G proteins, ion channels, and signaling molecules for neurotransmitter release and excitation/contraction coupling in smooth muscle cells is highly plastic (Lomax et al., 2005; Shi et al., 2005; Shi and Sarna, 2004 and 2000). For example, the expression of the pore-forming subunit of L-type Ca²⁺ channels is suppressed in colonic inflammation and this contributes to the suppression of RPCs and tone (Liu et al., 2001; Shi et al., 2005). More important for the design of promotility drugs, the expression of key signaling molecules for excitation-contraction coupling can be enhanced by neurotransmitters, such as VIP and ACh (Shi et al., 2006; Shi and Sarna 2006), and presumably by sustained exposure to other pharmacological compounds that can stimulate appropriate transcription factors in circular smooth muscle cells. The treatment of human colonic circular smooth muscle cells with VIP induces the gene expression of the α_{1C} submit of L-type of Ca²⁺ channels, while the treatment of these cells with ACh enhances the expression of MLC₂₀. In both cases, the contractile response of muscle strips incubated with VIP or ACh is enhanced when compared to strips incubated with medium only. VIP induces the gene expression of α_{1C} by activating adenylyl cyclase, synthesizing cAMP and phosphorylating PKA, which phosphorylates transcription factor cAMP response element binding protein (CREB) (64). Thus, agents that increase cAMP resulting in the increased expression of α1C will lead to a sustained contractility. The promoter (α1C1b) of α1C gene that is 5′ to exon 1b has two binding sites for CREB (Shi et al., 2006; Shi et al., 2005). These findings indicate that the impairment of ACh release from the enteric neurons may be compensated by enhanced-expression of key signaling molecules for excitation-contraction coupling in smooth muscle cells. The targeting of specific molecules for excitation/contraction coupling in smooth muscle cells may allow for selective enhancement of the three types of gut contractions in different organs.

B Other Disorders Involving Smooth Muscle Cells

It is contemplated the present invention can be used to alter the contractility of different smooth muscle cells through the modulation of α_{1C} expression. Other embodiments concern the use of α_{1C} modulators in treatment modalities for diseases that involve smooth muscle cells other than gut smooth muscle cells.

It is contemplated that diseases and conditions that involve other smooth muscle cells can also be addressed by the present invention. Smooth muscle cells include visceral and vascular smooth muscle cells. In certain embodiments, it is contemplated that diseases and conditions involving contractility of the vasculature are included as part of the invention. It is contemplated that such diseases and conditions include, but are not limited to, hypertension, thromboses, and coronary artery disease. In other embodiments, it is contemplated that diseases and conditions involving smooth muscle cells of the trachea, uterus, or bladder can be treated. Such diseases and conditions of the trachea include, but are not limited to, gastroesophageal disease, relapsing polychondtritis, stenosis, tracheitis, and Wegener's granulomatosis. Diseases and conditions of the uterus include, but are not limited to, endometriosis, fibroids or tumors, polyps, and uterine prolapse. Diseases and conditions of the bladder include, but are not limited to, bladder control conditions (incontinence generally, bladder incontinence, overactive bladder, overflow incontinence, mixed incontinence), cystitis, and urinary tract infections.

Other embodiments apply to the treatment of gallbladder diseases and conditions, including gallbladder disease (also biliary disease), cholecystitis, cholelithiasis, and cholangitis.

### III Modulators of α_{1C} Subunit Expression

The present invention concerns agents that alter the expression of the α_{1C} subunit of L-type calcium channels. In certain embodiments the present invention concerns neurotransmitters that operate in and around smooth muscle cells, particularly gut smooth muscle cells.

A Peptide Neurotransmitters

The peptide neurotransmitters VIP and PACAP (see McConalogue et al., 1994 for a review) are contemplated as part of the invention because they induce expression of the α_{1C} subunit. While the focus of discussion is on VIP and PACAP, it is contemplated that embodiments discussed in the application apply to any α_{1C} modulator. VIP is a 28 amino acid polypeptide hormone whose sequence is identical in many mammals. An exemplary amino acid sequence of VIP is provided in SEQ ID NO:1. An example of the amino acid sequence of human PACAP-38 is provided in SEQ ID NO:2, though it is contemplated that the term “PACAP” covers PACAP-27, which is exemplified in SEQ ID NO:3.

VIP and PACAP are members of a super family of structurally related peptide hormones that include secretin, glucagon, and growth hormone releasing factor. VIP is a 28 amino acid peptide whose role as an inhibitory neurotransmitter of non-adrenergic non-cholinergic neurons in the gut has been examined in-depth (Murthy et al., 1996; Grider et al., 1994; Grider and Rivier, 1990; Lomax and Furness, 2000; Schultzberg et al., 1978; Talmage and Mawe, 1993; Zafirov et al., 1985). PACAP occurs in 27-(PACAP27) and 38-(PACAP38) forms and bears partial homology to VIP. Both VIP and PACAP peptides are distributed widely in enteric inhibitory motor neurons that directly innervate the circular smooth muscle cells (Harmar et al., 2004; Uemura et al., 1998; Furness et al., 1990; Pluja et al., 2000; Ekblad, 1999; Grider, 2003).

Three VIP/PACAP receptors have been cloned: VPAC₁, VPAC₂, and PAC₁, (Lutz et al., 1993; Pisegna and Wank, 1993; Svoboda et al., 1993). The PAC₁, receptor is relatively selective for PACAP. By contrast, VPAC₁, and VPAC₂ receptors exhibit equal affinity to VIP and PACAP (Harmar et al., 1998). All three receptors are present on circular muscle cells in the gut. However, their roles in gene transcription in these cells have not been investigated.

PAC₁ receptors have been reported to be coupled to adenylate cyclase and phospholipase C through guanine-nucleotide-binding proteins Gαs and Gα_{q/ll} respectively (Rawlings and Hezareh, 1996). VPAC₁, and VPAC₂ receptors are coupled primarily to adenylate cyclase, but in some cells they have been reported to couple also to phospholipase C (Sreedharan et al., 1994; Xia et al., 1997). These receptors may have additional direct or indirect effectors, e.g. PACAP-induced synaptic current in Drosophila is mediated by co-activation of the low molecular weight G protein kinase Ras and cAMP signaling pathways (Zhong, 1995). Overall then, VIP and PACAP may concurrently activate a number of signaling pathways, including cAMP/PKA through the activation of adenylate cyclase, Ca²⁺/PKC pathway through the activation of phospholipase C, calcium/calmodulin-dependent protein kinase II (CaMKII), and MAP kinase pathways thorough the activation of small G proteins (Harmar et al., 1998). These signaling pathways may also have cross-talk. For example, cAMP and PKA may modulate the activation of MAPK signaling through their actions on Ras and Raf-1.

Moreover, agonists and antagonists of both VIP and PACAP receptors in gut smooth muscle cells are contemplated for use in the invention. See Forrsmann et al., 1998, which is hereby incorporated by reference and Table 1.

TABLE 1
Nomenclature of receptors for PACAP and VIP

| Receptor subtype | | | Human | | |
|---|---|---|---|---|---|
| IUPHAR | Previous | Gene name | chromosome | | Selective |
| nomenclature | nomenclature | (HUGO) | location | Selective agonists | antagonist |
| PAC₁ | PACAP type I | ADCYAP1R1 | 7p14 | Maxadilan | PACAP(6- |
| | | | | | 38)^{a} |
| | PVR1 | | | | |
| VPAC₁ | VIP | VIPR1 | 3p22 | [Arg¹⁶]chicken | [Ac-His¹, D- |
| | | | | secretin^{b} | Phe², Lys¹⁵, |
| | | | | | Arg¹⁶] |
| | VIP₁ | | | [K¹⁵R¹⁶L²⁷]VIP(1- | VIP(3- |
| | | | | 7)GRF(8-27)-NH₂ | 7)GRF(8- |
| | | | | | 27)-NH₂ |
| | PACAP type | | | | |
| | II | | | | |
| | PVR2 | | | | |
| VPAC₂ | VIP₂ | VIPR2 | 7q36.3 | Ro 25-1553 | — |
| | PACAP-3 | | | Ro 25-1392 | |
| | PVR3 | | | | |

^{a}Displays significant affinity for VPAC₂ receptors.
^{b}Selective only in rodent tissues (e.g., brain) that do not express the secretin receptor.

Agonists of VIP and PACAP include, but are not limited to, a lipophilic VIP analogue (stearyl, norleucine 17-VIP [SNV]) that has neuroprotective properties and Ro25-1553. See Gozes et al., 1996; Wang et al., 1999, both of which are incorporated by reference.

Antagonists of VIP and PACAP receptors include, but are not limited to, 1) [4-Cl-D-Phe6,Leu17]-VIP, 2) [Ac-Tyr1,D-Phe2]-Growth Hormone Releasing Factor 1-29 amide, 3) VIP₁₀-28 (see Grider, 2003, which is hereby incorporated by reference), 4) Neurotensin6-11-VIP7-28 (hybrid peptide antagonist), and 5) Stearyl, Norleucine17-hybrid antagonist (SNH).

Other specific agonists and antagonists of VPAC₁ include, but are not limited to, ((Lys¹⁵,Arg¹⁶,Leu²⁷)VIP(3-7)-GRF(8-27) and (Ac-His¹,D-Phe²,Lys¹⁵,Arg¹⁶ Leu²⁷)VIP(3-7)-GRF(8-27) respectively); VPAC₂ (Ro 25-1392 Ac-(Glu⁸,O—CH3-Tr¹⁰,Lys¹²,Nle¹⁷Aa¹⁹,Asp²⁵,Leu²⁶,Lys^{27,28})-VIP cyclo(21-25)) and PAC₁ (Maxadilan and PACAP(6-38), respectively.

In some embodiments of the invention, agonists will be used at concentrations of 0.1, 1 and 10 nM and the antagonists at 0.01, 0.1 and 1 μM to obtain excitatory and inhibitory concentration-response curves, respectively. These concentrations have been reported in the literature to be effective in blocking or stimulating these receptors (Hannar et al., 1998; Rawlings and Hezareli, 1996; Abad et al., 2003; Delgado et al., 1998).

1 Proteinaceous Compositions

In certain embodiments, the present invention concerns compositions comprising at least one proteinaceous molecule, such as a peptide hormone like VIP or PACAP. As used herein, a “proteinaceous molecule,” “proteinaceous composition,” “proteinaceous compound,” “proteinaceous chain” or “proteinaceous material” generally refers to a peptide (between 4 and 100 amino acids in length), but it also includes a protein of greater than about 200 amino acids or the fill length endogenous sequence translated from a gene; a polypeptide of greater than about 100 amino acids; and/or a peptide of from about 3 to about 100 amino acids. All the “proteinaceous” terms described above may be used interchangeably herein.

In preferred embodiments, a proteinaceous compound will have fewer than 50 amino acid residues. It is contemplated that in certain embodiments the size of the proteinaceous molecule may be, be at least, or be at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or greater amino acid residues in length, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 2 below.

TABLE 2
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (World Wide Web at ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific protein, polypeptide, or peptide composition that has been subjected to fractionation to remove various other proteins, polypeptides, peptides and other materials, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout a procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

a. Functional Aspects

When the present application refers to the function or activity of VIP or PACAP, it is meant that the molecule in question has the ability to induce transcription of the α_{1C} subunit of the L-type calcium channel in gut smooth muscle cells. Other phenotypes that may be considered to be associated with the VIP or PACAP gene product are the ability to promote relaxation of smooth muscle cells in a pharmacological (non-genomic) manner or to be involved in homeostasis of gut smooth muscle cells, or to be involved in the prevention or treatment of gastrointestinal motility diseases or disorders. Determination of which molecules possess this activity may be achieved using assays familiar to those of skill in the art. For example, assays that measure α_{1C} gene expression levels can identify, by virtue of an increased level of gene expression, those molecules having a VIP or PACAP activity or function.

On the other hand, when the present invention refers to the function or activity of a “VIP or PACAP receptor agonist” one of ordinary skill in the art would further understand that this includes, for example, the ability to specifically or competitively bind a VIP or PACAP receptor or an ability to promote gene expression of the α_{1C} subunit. Thus, it is specifically contemplated that a VIP or PACAP agonist has the ability to act as VIP or PACAP. Determination of which molecules are suitable agonists of a VIP or PACAP receptor may be achieved using assays familiar to those of skill in the art—some of which are disclosed herein.

Moreover, in some embodiments there is a VIP or PACAP receptor antagonist, which one of ordinary skill in the art would understand refers to a compound that has the ability to specifically bind a VIP or PACAP receptor and prevent binding of VIP or PACAP to that receptor. It will also be understood that such an antagonist will prevent VIP or PACAP induction of gene expression of the α_{1C} subunit.

b. Variants of VIP and PACAP

In some embodiments, VIP and/or PACAP variants (also referred to as analogs) can be used in methods of the invention, so long as the variants are capable of acting as good as, if not better than, the nonvariant VIP or PACAP. The variants may have changes in the actual sequence of the amino acid residue, for example, substitutional, insertional or deletion variants. Alternatively, the variants may have chemical modifications such that the chemical structure of the protein is altered in a different way other than adding, substituting, or deleting one or more amino acid residues.

Deletion variants lack one or more residues of the native protein that are not essential for function or immunogenic activity. Insertional mutants typically involve the addition of material at a non-terminal or terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Other variants may be analogs, a number of which are well known. See U.S. Pat. No. 4,939,224, U.S. Pat. No. 4,866,039, U.S. Pat. No. 4,605,641, Couvineau et al., 1984, Beyerman et al., 1981, Takeyama et al., 1980, Gardner et al., 1980, Bodansky et al., 1978, all of which are incorporated herein by reference.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of a VIP or PACAP polypeptide or a VIP or PACAP analog provided the biological activity of the protein is maintained.

The term “functionally equivalent codon” is used herein to refer to codons that encode the same amino acid, such as the six codons for arginine or serine, and also refers to codons that encode biologically equivalent amino acids (see Table 3, below).

TABLE 3
Codon Table

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, binding sites on receptors. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 2 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure, See e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of VIP or PACAP, but with altered and even improved characteristics.

c. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

d. Protein Purification

It may be desirable to purify VIP, PACAP, or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

e. Synthetic Peptides

The compositions of the invention may include a synthetic peptide, including one that is modified to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides which include all L-amino acids, all D-amino acids, or a mixture thereof. The use of D-amino acids may confer additional resistance to proteases naturally found within the human body and are less immunogenic and can therefore be expected to have longer biological half lives.

The present invention describes neurotransmitter peptides for use in various embodiments of the present invention. For example, specific peptides are assayed for their abilities to induce gene expression of the α_{1C} subunit of the L-type calcium channel. In specific embodiments that the peptides are relatively small in size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

The compositions of the invention may include a peptide modified to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides which include all L-amino acids, all D-amino acids, or a mixture thereof. The use of D-amino acids may confer additional resistance to proteases naturally found within the human body and are less immunogenic and can therefore be expected to have longer biological half lives.

f. In Vitro Protein Production

In addition to the purification methods provided in the examples, general procedures for in vitro protein production are discussed. Following transduction with a viral vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshney, 1992).

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses autologous B lymphocyte cell lines, which are transfected with a viral vector that expresses an immunogen product, and more specifically, a protein having immunogenic activity. Other examples of mammalian host cell lines include Vero and HeLa cells, other B- and T-cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk−, hgprt− or aprt− cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

g. Nucleic Acids

In accordance with the objects of the present invention, a polynucleotide that encodes a protein, polypeptide, peptide, or functional equivalent thereof, may be used to generate recombinant DNA molecules that direct the expression of these proteinaceous compounds in appropriate host cells.

Due to the inherent degeneracy of the genetic code, other DNA sequences that encode substantially the same or a functionally equivalent amino acid sequence, may be used in the practice of the invention of the cloning and expression of the protein. Such DNA sequences include those capable of hybridizing to the sequences or their complementary sequences under stringent conditions. In one embodiment, the phrase “stringent conditions” as used herein refers to those hybridizing conditions that (1) employ low ionic strength and high temperature for washing, for example, 0.015 M NaCl/0.0015 M sodium citrate/0.1% SDS at 50° C.; (2) employ during hybridization a denaturing agent such as formamide, for example, 50% (vol/vol) formamide with a 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or (3) employ 50% formamide, 5×SSC (0.75 M NaCl, 0.075 M Sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS.

Altered DNA sequences that may be used in accordance with the invention include deletions, additions or substitutions of different nucleotide residues resulting in a sequence that encodes the same or a functionally equivalent fusion gene product. The gene product itself may contain deletions, additions or substitutions of amino acid residues within a sequence, which result in a silent change thus producing a functionally equivalent protein. Such amino acid substitutions may be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine, histidine and arginine; amino acids with uncharged polar head groups having similar hydrophilicity values include the following: glycine, asparagine, glutamine, serine, threonine, tyrosine; and amino acids with nonpolar head groups include alanine, valine, isoleucine, leucine, phenylalanine, proline, methionine, tryptophan.

The DNA sequences of the invention may be engineered in order to alter a coding sequence for a variety of ends, including but not limited to, alterations which modify processing and expression of the gene product. For example, mutations may be introduced using techniques which are well known in the art, e.g., site-directed mutagenesis, to insert new restriction sites, to alter glycosylation patterns, phosphorylation, etc.

In an alternate embodiment of the invention, the coding sequence of the protein could be synthesized in whole or in part, using chemical methods well known in the art. (See, for example, Caruthers et al., 1980; Crea and Horn, 1980; and Chow and Kempe, 1981). For example, active domains of moieties can be synthesized by solid phase techniques, cleaved from the resin, and purified by preparative high performance liquid chromatography followed by chemical linkage to form a chimeric protein. (e.g., see Creighton, 1983a). The composition of the synthetic peptides may be confirmed by amino acid analysis or sequencing (e.g., the Edman degradation procedure; see Creighton, 1983b). Alternatively, protein produced by synthetic or recombinant methods may be conjugated by chemical linkers to another protein according to methods well known in the art (Brinkmann and Pastan, 1994).

In order to express a biologically active protein, the nucleotide sequence coding for a protein, or a functional equivalent, is inserted into an appropriate expression vector, i.e., a vector which contains the necessary elements for the transcription and translation of the inserted coding sequence. The gene products as well as host cells or cell lines transfected or transformed with recombinant expression vectors can be used for a variety of purposes. These include but are not limited to generating antibodies (i.e., monoclonal or polyclonal) that bind to epitopes of the proteins to facilitate their purification.

Methods that are well known to those skilled in the art can be used to construct expression vectors containing the protein coding sequence and appropriate transcriptional/translational control signals. These methods include in vitro recombinant DNA techniques, synthetic techniques and in vivo recombination/genetic recombination. See, for example, the techniques described in Sambrook et al. (2001) and Ausubel et al. (1996).

A variety of host-expression vector systems may be utilized to express the protein coding sequence. These include but are not limited to microorganisms such as bacteria transformed with recombinant bacteriophage DNA, plasmid DNA or cosmid DNA expression vectors containing the protein coding sequence; yeast transformed with recombinant yeast expression vectors containing the protein coding sequence; insect cell systems infected with recombinant virus expression vectors (e.g., baculovirus) containing the protein coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the protein coding sequence; or animal cell systems. It should be noted that since most apoptosis-inducing proteins cause programmed cell death in mammalian cells, it is preferred that the protein of the invention be expressed in prokaryotic or lower eukaryotic cells.

The expression elements of each system vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter; cytomegalovirus promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the DNA, SV40-, BPV- and EBV-based vectors may be used with an appropriate selectable marker.

An alternative expression system which could be used to express protein is an insect system. In one such system, Autographa californica nuclear polyhidrosis virus (AcNPV) is used as a vector to express foreign genes. The virus grows in Spodoptera frugiperda cells. The protein coding sequence may be cloned into non-essential regions (for example the polyhedrin gene) of the virus and placed under control of an AcNPV promoter (for example the polyhedrin promoter). (e.g., see Smith et al., 1983; U.S. Pat. No. 4,215,051).

Other plant-based systems exist for protein production purposes, such as those discussed in U.S. Pat. No. 6,136,320.

Specific initiation signals may also be required for efficient translation of the inserted protein coding sequence. These signals include the ATG initiation codon and adjacent sequences. The efficiency of expression may be enhanced by the inclusion of appropriate transcription enhancer elements, transcription terminators, etc. (see Bittner et al., 1987).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the protein. To this end, eukaryotic host cells which possess the cellular machinery for proper processing of the primary transcript, glycosylation, and phosphorylation of the protein may be used. Such mammalian host cells include but are not limited to CHO, VERO, BHK, HeLa, COS, MDCK, 293, W138, and the like.

For long-term, high-yield production of recombinant chimeric proteins, stable expression is preferred. For example, cell lines which stably express the chimeric protein may be engineered. Rather than using expression vectors which contain viral originals of replication, host cells can be transformed with a coding sequence controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including but not limited to the herpes simplex virus thymidine kinase (Wigler et al., 1977), hypoxanthine-guanine phosphoribosyltransferase (Szybalski and Szybalski, 1962), and adenine phosphoribosyltransferase (Lowy et al., 1980) genes can be employed in tk−, hgprt− or aprt− cells, respectively. Also, antimetabolite resistance can be used as the basis of selection for dhfr, which confers resistance to methotrexate (Wigler et al., 1980; O'Hare et al., 1981); gpt, which confers resistance to mycophenolic acid (Mulligan and Berg, 1981); neo, which confers resistance to the aminoglycoside G-418 (Colbere-Garapin et al., 1981); and hygro, which confers resistance to hygromycin (Santerre et al., 1984) genes. Additional selectable genes have been described, namely trpB, which allows cells to utilize indole in place of tryptophan; hisD, which allows cells to utilize histinol in place of histidine (Hartman and Mulligan, 1988); and ODC (ornithine decarboxylase) which confers resistance to the omithine decarboxylase inhibitor, 2-(difluoromethyl)-DL-ornithine, DFMO (McConlogue, 1987).

In a further embodiment of the invention, the gene construct may be entrapped in a liposome or lipid formulation (Ghosh and Bachhawat, 1991). Also contemplated is a gene construct complexed with Lipofectamine (Gibco BRL). Recent advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. Beneficial characteristics of these lipid structures include a positive colloidal stabilization by cholesterol, two dimensional DNA packing and increased serum stability.

B Other Neurotransmitters

Like VIP and PACAP, norepinephrine is a neurotransmitter that has also been show to induce expression of the α_{1C} subunit. Norepinephrine is also considered a hormone. Agonists and antagonists of norepinephrine receptors can be used in methods of the invention. Agonists of norepinephrine include, but are not limited to, desipramine, mazindol, and nortriptyline, epinephrine, methoxamine, phenylephrine, clonidine, dobutamine, isoproterenol, albuterol, isoetharine, terbutaline. Antagonists that are contemplated for use include, but are not limited to, doxazosin, prazosin, tamsulosin, and terazosin, yohimbine, beta-blockers (used to treat hypertension and heart disease) such as atenolol and metoprolol, and propranolol.

C Signalling Pathway

CREB is member of a large family of structurally related transcription factors that include c-Jun and c-Fos (Shaywitz and Greenberg, 1999). The members of this family, named bZIP family, share a dimerization domain with a leucine zipper motif and a DNA-binding domain rich in basic residues (lysines and arginines). CREB proteins specifically recognize the palindromic promoter site 5′-TGACGTCA-3′ (Montminy, 1997). Many CREB binding sites are variations of this consensus sequence but, in most cases, the core sequence CGTCA is maintained (Lonze and Ginty, 2002). Two other gene products largely homologous to CREB have also been characterized, activating transcription factor-1 (ATF-1) (Hai et al., 1989) and cAMP response element modulator (CREM) (Foulkes et al., 1991). These three CREB family members bind to CRE as homo- and heterodimers (Yamamoto et al., 1988). The ratio between homo- and hetero-dimers is cell-type dependent and determines CREB's transcriptional activity because homodimers have a longer half-life than the heterodimers.

A diverse array of stimuli, including peptide hormones, growth factors and neurotransmitters activate transcription of CREB target genes. As noted in a previous paragraph, the activation of CREB is mediated by a variety of kinases that phosphorylate CREB at Ser 133 (Gonzalez and Montiminy, 1989). Following the phosphorylation of Ser 133, CREB binding protein (CBP) is recruited to CREB and hence to the CRE sequence in the promoter. The recruitment of this co-activator has two functions 1) CBP has the ability to bind basal transcriptional components and hence stabilize the pre-initiation complex that forms at the promoter (Kwok et al., 1994) and 2) CBP, via its endogenous histone acetyltransferase (HAT) activity (Bannister and Kouzarides, 1996), facilitates the unraveling of chromatin to increase the accessibility of the local chromatin to transcriptional machinery complexes (Lonze and Ginty, 2002). Thus, the transcription of CREB target genes may be modulated by cAMP-dependent and cAMP-independent mechanisms.

CREB has been reported to regulate well over 100 genes that control neurotransmission, cell structure, cell proliferation, cell differentiation, and adaptive responses (Lonze and Ginty, 2002). In spite of the large number of genes that have been reported to be regulated by CREB, the regulation of the pore-performing α_{1C} subunit of L-type Ca²⁺ channels in smooth muscle cells by CREB has not been investigated. Fan et al. (2002) identified a 27 bp cis-acting sequence on the promoter of Caᵥ1.2a channels that are expressed in neonatal human and rat cardiac myocytes and rat vascular smooth muscle cells that was essential for their expression in response to α-adrenergic receptor agonist phenylephrine. This sequence contains c-Ets and AP-1 binding sites, indicating that synergistic transcriptional activation of these two transcription factors was required for the expression of Caᵥ1.2a channels in cardiac myocytes and vascular smooth muscle cell line PAC1. Maki et al. (Maki et al., 1996) found that P-adrenergic agonist isoproterenol or 8-bromo-cAMP produced a transient increase in Caᵥ1.2a mRNA accompanied by an increase of about 40% in peak current density in neonatal rat ventricular myocytes. However, these results have not been reproduced in adult cardiac myocytes yet.

CREB-induced gene expression is cell type- and ligand-specific, even though the only essential requirement for the activation of CREB is phosphorylation at Ser 133 (Silva et al., 1998; Cambi et al., 1989). The specificity in response is achieved in part by the activation of different co-factors for gene transcription in response to different stimuli in the same cell or the same stimulus in different cells. Identification of the various signaling pathways that are activated in response to a specific stimulus in a cell can be informative. In addition, the time course of the target gene expression may also depend upon the continued generation of key second messengers, such as cAMP. In some cells, cAMP generation in response to a stimulus has been reported to be transient (Lonze and Ginty, 2002; Chen et al., 1999), but the data in the Examples show that in HCCSMC, VIP elevated cAMP levels in HCCSMC for at least 24 h.

MAPK cascades are evolutionarily conserved in eukaryotes and play a key role in multiple cellular functions, including cell differentiation, cell division, cell movement, apoptosis and cell contraction (Schaeffer and Weber, 1999; Khokhatchev et al., 1998; English et al., 1999; Houslay and Kolch, 2000). The roles of MAPKs in the regulation of α_{1C} gene expression by VIP or PACAP have not been investigated. However, the data in the Examples showed that VIP time-dependently dephosphorylates the three MAPKs. It is, therefore, likely that one or more of these cascades may modulate the transcription of α_{1C} gene.

Ligands that bind to G protein coupled receptors stimulate MAPKs by multiple mechanisms (English et al., 1999). The activation of G proteins may enhance or suppress the phosphorylation of ERK (½ by increasing the activity of PKA or through interaction with small GTPases of the Ras and Rho families (Houslay and Kolch, 2000). The elevation of cAMP in fibroblasts and vascular smooth muscle cells induces inhibition of ERK (activation by acting downstream of Ras but upstream of Raf-1 (Burgerfing et al., 1993; Russell et al., 1994). PKA phosphorylates Raf-1 on ser 43 to reduce its affinity to Ras/GTP and hence the constitutive activation of ERKs by Ras.

PKC also activates the Ras/Raf pathway (Sweatt, 2001). In addition, a family of phorbol ester-binding Ras/Raf guanine neucleotide exchange factors (GEFs) may also allow the second messenger diacylglycerol (DAG) to activate ERKs independent of PKC. It has not previously been determined which pathways are activated by VIP/PACAP and how these pathways interact to enhance the expression of α_{1C} gene.

The downstream targets of MAPKs include several transcription factors, such as CREB, AP-1 and Elk-2. The term AP-1 (activating protein-1) refers to a family of dimeric transcription factors comprised of Jun, Fos and ATF (activating transcription factor) subunits that bind to a common binding motif (AP-1 binding site) (Vogt and Bos, 1990; Angel and Karin, 1991). The potential binding of AP-1 transcription factors whose components are phosphorylated by MAPKs provides the link for MAPKs to regulate α_{1C} gene whose promoter has two CRE elements. IV Methods of Treatment

In certain embodiments of the present invention, methods involving delivery of a peptide or an expression construct encoding the peptide are contemplated. In some embodiments, the method is directed to delivery of a neurotransmitter over a sustained period of time and/or is delivered in a way so as to provide a gut smooth muscle cell with a relatively low dosage. In certain aspects, an agent can be administered that elevates cAMP levels resulting in the sustained expression of α_{1C}. Such methods are employed to achieve sustained gene expression of the α_{1C} subunit of the L-type calcium channel.

A Administration

In certain specific embodiments, it is desired to promote or induce expression of the α_{1C} subunit, particularly for at least two hours using the methods and compositions of the present invention. The routes of administration can vary depending on the particular indication being addressed, and it is contemplated that administration may be intradermal (including transdermal), subcutaneous, regional, parenteral, intrarectal, intravenous, intramuscular, systemic, and oral administration and formulation.

In certain embodiments, a therapeutic composition utilizes transdermal, oral, percutaneous, or intrarectal administration. Such routes of administration may be systemic or intermittent. In addition, single administrations are also contemplated. These routes of administration are well known to those of skill in the art, as illustrated by, for example, U.S. Pat. Nos. 4,889,721 (percutaneous), 4,781,924 (transdermal), 4,908,027 (transdermal), 6,007,837 (transdermal agent production), 5,990,179 (electrotransport), 4,942,037 (transdermal), EP 0 072 251 (transdermal), and 4,624,665 (transdermal), all of which are incorporated by reference. Moreover, in the therapy of various diseases, transdermal therapeutic systems (TTS) have been introduced. See, for example, U.S. Pat. No. 6,521,250, which is hereby incorporated by reference.

Continuous perfusion or administration of an agent is also contemplated. The amount of construct or peptide delivered in continuous perfusion can be determined by the amount of uptake that is desirable. This can be accomplished by intravenous administration.

Delivery via syringe or catheterization is used in some embodiments. Such continuous administration may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the administration occurs.

Treatment regimens may vary as well, and often depend on severity of symptoms, disease progression, and health and age of the patient. Obviously, certain types of disorders will require more aggressive treatment, while at the same time, certain patients cannot tolerate more taxing protocols. The clinician will be best suited to make such decisions based on the known efficacy and toxicity (if any) of the therapeutic formulations.

In certain embodiments, treatment includes other traditional treatments for gastrointestinal disorders, including a regimen of antibiotics, laxatives, anti-diarrheals, and/or surgery.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may be described in terms of amount/kg/day or amount/kg for a course of treatment.

Agents may be administered to a patient in concentrations of about or of at least about 0.01. 0.05, 0.1, 0.2, 0.3, 0.4, 0.5, 0.6, 0.7, 0.8, 0.9, 1.0, 2.0, 3.0, 4.0, 5.0, 6.0, 7.0, 8.0. 9.0, 10, 15, 20, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 or more ng/ml or 0.01. 0.05, 0.1, 0.2, 0.3, 0.4, 0.5, 0.6, 0.7, 0.8, 0.9, 1.0, 2.0, 3.0, 4.0, 5.0, 6.0, 7.0, 8.0. 9.0, 10, 15, 20, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 nM.

B Compositions and Formulations

The pharmaceutical compositions disclosed herein may alternatively be administered parenterally, intrarectally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of agents may be delivered by syringe or any other method used for injection of a solution, as long as the expression construct can pass through the particular gauge of needle required for injection. A novel needleless injection system has recently been described (U.S. Pat. No. 5,846,233) as well as a syringe system for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the some methods of preparation are vacuum-drying and freeze-drying techniques which yield a powder of the active ingredient plus any additional desired ingredient from a previously sterile-filtered solution thereof.

Other formulations include those appropriate for aerosolization and inhalation, which may or may not be used for the treatment of respiratory diseases or conditions (including asthma and reactive airway disease). Such formulations may involve formulating a particular particle size or the contents of the inactive ingredients in the composition. Pulmonary drug delivery can implemented by different approaches, including liquid nebulizers, aerosol-based metered dose inhalers (MDI's), and dry powder dispersion devices. Such methods and compositions are well known to those of skill in the art, as indicated by U.S. Pat. Nos. 6,797,258, 6,794,357, 6,737,045, and 6,488,953, all of which are incorporated by reference.

Mucosal and buccal delivery and formulation are also specifically contemplated. Examples are readily available, as described in U.S. Pat. Nos. 6,488,953, 6,284,262, 5,863,555, and 5,726,154, which are hereby incorporated by reference.

Additional formulations suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

It is particularly contemplated that agents may be formulated and/or delivered for slow release and/or sustained release. Such formulations and delivery methods are well known to those of skill in the art, and they include transdermal formulations. Examples of slow and sustained release can be found in U.S. Pat. Nos. 6,074,673, 6,045,824, 5,055,307, 4,808,416, and 4,503,031, which are hereby incorporated by reference. In certain embodiments, a sustained release suppository is contemplated. This formulation is well known as shown, for example, in U.S. Pat. Nos. 5,500,221, 5,436,009, and 3,962,436, which are hereby incorporated by reference.

1 Lipid Compositions

In certain embodiments, the present invention employs a composition comprising one or more lipids associated with at least one agent. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

In certain embodiments, a lipid component of a composition can be uncharged or primarily uncharged or it may be charged.

In certain embodiments, a lipid composition may comprise about 1%, about 2%, about 3%, about 4% about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. Thus, it is contemplated that lipid compositions of the present invention may comprise any of the lipids, lipid types or other components in any combination or percentage range.

A lipid may be comprised in an emulsion. Methods for preparing lipid emulsions and adding additional components are well known in the art (e.g., Modern Pharmaceutics, 1990, incorporated herein by reference).

A lipid may be comprised in a micelle. A micelle may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference).

In particular embodiments, a lipid comprises a liposome. A “liposome” is a generic term encompassing a variety of single and multilamellar lipid vehicles formed by the generation of enclosed lipid bilayers or aggregates.

In another liposome formulation, an amphipathic vehicle called a solvent dilution microcarrier (SDMC) enables integration of particular molecules into the bi-layer of the lipid vehicle (U.S. Pat. No. 5,879,703). Of course, any other methods of liposome preparation can be used by the skilled artisan to obtain a desired liposome formulation in the present invention.

### V Screening Methods

The present invention further comprises methods for identifying candidate therapeutic agents for gastrointestinal motility disorders by identifying compounds that modulate the gene expression of the α_{1C} subunit of the L-type calcium channel. These assays may comprise random screening of large libraries of candidate substances; alternatively, the assays may be used to focus on particular classes of compounds selected with an eye towards structural attributes that are believed to make them more likely to modulate the gene expression of the α_{1C} subunit, for example, by modifying something in the pathway leading to gene expression. Alternatively, the candidate substance may be one that is a VIP or PACAP analog or an agonist or antagonist of a VIP or PACAP receptor. Thus, it may have the function of VIP or PACAP or an antagonist thereof.

By gene expression, it is meant that one may assay for a measurable effect on the level of transcript and/or protein. To identify a possible candidate therapeutic substance, one generally will determine the level of gene expression in the presence and absence of the candidate substance, wherein a candidate therapeutic substance is defined as any substance that alters gene expression of α_{1C} subunit. For example, a method generally comprises: a) contacting a cell with the candidate agent; b) assaying gene expression of the α_{1C} subunit of the L-type calcium channel in the cell; c) comparing the levels of gene expression of the α_{1C} subunit in the presence and absence of the candidate agent, wherein a difference between the measured characteristics indicates that the candidate agent is, indeed, a candidate therapeutic compound.

Assays may be conducted in cell free systems, in isolated cells, or in organisms including transgenic animals. In a cell free system, an α_{1C} subunit promoter is contacted with the candidate agent and expression levels evaluation with or without the candidate agent.

It will, of course, be understood that all the screening methods of the present invention are useful in themselves notwithstanding the fact that effective candidates may not be found. The invention provides methods for screening for such candidates, not solely methods of finding them. Moreover, aspects of the invention further provide for manufacturing a candidate agent that affects the level of α_{1C} subunit expression levels. Such a candidate agent may be provided to an animal model for further testing and eventually to a patient to evaluate its therapeutic efficacy.

A Candidate Substance

As used herein the term “candidate substance” or “candidate agent” refers to any molecule that may be a “modulator” of α_{1C} gene expression levels, transcript or protein, unless otherwise indicated. A modulator may be a “α_{1C} expression inhibitor,” which is a compound that overall effects an inhibition of α_{1C} gene expression. A modulator may be a “α_{1C} expression enhancer,” which enhances or increases α_{1C} gene expression. Any modulator described in methods and compositions herein may be an inhibitor or an enhancer.

The candidate substance may be a protein or fragment thereof, a small molecule, or even a nucleic acid molecule such as a siRNA. An example of pharmacological compounds will be compounds that are structurally related to VIP or PACAP, or a molecule that binds one or more receptors for VIP or PACAP, or those that affect CREB. For example, the cDNA sequence for CREB can be found in Genbank in Accession No. NM_{—}004379. An siRNA can be constructed with CREB as a target, such as those siRNA used herein.

Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include siRNA, antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

1 In Vitro Assays

A quick, inexpensive and easy assay to run is an in vitro assay. Such assays generally use isolated molecules, can be run quickly, and in large numbers, thereby increasing the amount of information obtainable in a short period of time. A variety of vessels may be used to run the assays, including test tubes, plates, dishes and other surfaces such as dipsticks or beads.

One example of a cell free assay is a DNA binding assay or in vitro transcription assay. The target may be either free in solution, fixed to a support, expressed in or on the surface of a cell. Other indirect assays may involve screening candidates for an ability to bind a protein in the pathway leading to induction of α_{1C} subunit expression as a result of VIP, PACAP or norepinephrine. A technique for high throughput screening of compounds is described in WO 84/03564. Large numbers of small peptide test compounds are synthesized on a solid substrate, such as plastic pins or some other surface. Bound polypeptide is detected by various methods.

cDNA libraries may also be constructed and/or used for screening purposes. Construction of such libraries and analysis of RNA using such libraries may be found in Sambrook et al. (2001); Maniatis et al. (1990); Efstratiadis et al (1976); Higuchi et al. (1976); Maniatis et al. (1976); Okayama et al. (1982); Gubler et al. (1983); Ko (1990); Patanjali et al. (1991); U.S. Patent Pub. 20030104468, each incorporated herein by reference.

The present methods and kits may be employed for high volume screening. A library of RNA or DNA, peptides, or polypeptides can be created using methods and compositions of the invention. This library may then be used in high throughput assays, including microarrays. Specifically contemplated by the present inventors are chip-based nucleic acid technologies such as those described by Hacia et al. (1996) and Shoemaker et al. (1996). Examples of arrays, their uses, and implementation of them can be found in U.S. Pat. Nos. 6,329,209, 6,329,140, 6,324,479, 6,322,971, 6,316,193, 6,309,823, 5,412,087, 5,445,934, and 5,744,305, which are herein incorporated by reference.

Microarrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for a product derived from transcription a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

The nucleic acid or analogue are attached to a solid support, which may be made from glass, plastic (e.g., polypropylene, nylon), polyacrylamide, nitrocellulose, or other materials. See Schena et al., 1995; DeRisi et al., 1996; Shalon et al., 1996. In principal, any type of array could be used, although, as will be recognized by those of skill in the art, very small arrays will be preferred because hybridization volumes will be smaller. Use of a biochip is also contemplated, which involves the hybridization of a labeled molecule or pool of molecules to the targets immobilized on the biochip.

2 Animal Models

Testing of compounds can be conducted in suitable animal models to evaluate efficacy as a treatment for a gastrointestinal motility disorder. Such an animal model would typically exhibit physiological symptoms of a gastrointestinal motility disorder or have such a disorder. Such animal models are well known to those of skill in the art.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The following materials and methods were used to generate the experimental data described or illustrated.

Primary cultures of HCCSMC: Human tissue was obtained from the descending and sigmoid colons with approval of the University of Texas Medical Branch Institutional Review Board from disease free margins of resected segments from patients undergoing surgery for colon cancer. The circular muscle layer was separated from the taenia coli and lamina propria with a tissue slicer. The circular muscle layer was collected in ice-cold HEPES buffer (in mM: 120 NaCl, 2.6 KH2S0₄, 4 KCl, 2 CaCl₂, 0.6 MgCl₂, 25 HEPES, 14 glucose and 2.1% essential amino acid mixture, pH 7.4). Two successive digestions with papain and collagenase, as described previously by Shi and Sarna, were used to disperse smooth muscle cells. The cells were cultured in RPMI 1640 (Gibco/Invitrogen, Carlsbad, Calif.) supplemented with 10% fetal bovine serum in the presence of 100 units/mL of penicillin G, 100 μg/mL of streptomycin sulfate and 0.25 μg/mL of amphoteracin B. The culture medium was changed every three days. Cells in passages 3 to 5 were used in all experiments. All cells were cultured in serum-free medium for at least 15 h prior to experiments to eliminate the possible effects of growth factors. Immunofluoresence imaging showed that more than 95% of the cultured cells stained for smooth muscle specific α-actin (data not shown). The cultures of colonic smooth muscle cells retain their contractile phenotype. Each experiment used cells or muscle strips from at least three different subjects.

Cytoplasmic and nuclear protein, and RNA extraction: Cytoplasmic and nuclear proteins were prepared as described earlier by Shi et al. Briefly, the cells were washed twice with phosphate-buffered saline solution and then with buffer A (in mM: 10 HEPES, 1.5 MgCl₂, 10 KCl, 0.5 dithiothreitol, 0.5 phenylmethylsulfrenyl fluoride [PMSF]; 10 ng/mL leupeptin, and 10 ng/mL aprotinin). The cells were resuspended in 25 μL of buffer A containing 0.25% NP-40 and incubated at 4° C. for 10 minutes. After centrifugation at 12,000 g for 10 minutes, the supernatant was collected as cytoplasmic extract. The nuclear pellet was resuspended in 20 μL of buffer C (in mM: 20 HEPES, 0.42 NaCl, 1.5 MgCl₂, 0.2 ethylenediaimetetraacetic acid (EDTA), 0.5 DTT, 0.5 PMSF; 24% glycerol, 10 ng/mL leupeptin, and 10 ng/mL aprotinin) and incubated on ice with frequent vortexing for 15 minutes. The samples were centrifuged at 14,000 g for 10 minutes, and the supernatant (nuclear extract) was transferred to a fresh tube with 5 volumes of buffer D (in mM: 20 HEPES, 0.2 EDTA, 50 KCl, 0.5 DTT, 0.5 PMSF; 20% glycerol, 10 ng/mL leupeptin, and 10 ng/mL aprotinin). The protein concentration was determined by using the Bio-Rad assay kit (Bio-Rad Laboratories, Hercules, Calif.).

Total RNA was extracted from cells using the Qiagen RNeasy kit (Qiagen, Valencia, Calif.) by following the manufacturer's protocol. The cDNAs were made using the Superscript First-Strand Synthesis System (Invitrogen, Carlsbad, Calif.).

Western immunoblotting. Each lane is loaded with 10 μg protein and size-separated on a 10% SDS polyacrylamide gel. After blotting, the nitrocellulose membranes are blocked with 10% non-fat dried milk at 4° C. overnight. The blots are incubated with appropriate dilution of the antibody at 4° C. overnight and appropriate dilution of designated secondary antibody at room temperature for 1 h. β-actin is used as internal control.

Quantitive real-time-polymerase chain reaction (Q-PCR). Q-PCR assay is employed to determine gene expression using TaqMan technology on Applied Biosystems 7000 sequence detection system (UTMB Real-time PCR Core Facility). Applied Biosystems Assays-By-Design containing a 20× assay mix of primers and TaqMan MGB probes (FAMTM dye-labeled) are used for a target gene and the endogenous control, human 18s RNA. These assays were designed using primers that span exon-exon junctions so as not to detect genomic DNA. All primer and probe sequences were searched against the Celera data base to confirm specificity. The primer and probe sequences used were as follows: human Caᵥ1.2 channel oα_{1C} intermediate form (Caᵥ1.2b) probe spanning exon 1b and exon 2, CACCAAGGTTCCAACTAT (SEQ ID NO:4): forward primer, CCATGGTCAATGAGAATACGAGGAT (SEQ ID NO:5): reverse primer, GCCGCATTGGCATTCATGTT (SEQ ID NO:6). Human Caᵥ1.2 channel α_{1C} long form (Caᵥ1.2a) probe spanning exon 1a and exon 2: CCCATAGTTGGAACACCTC (SEQ ID NO:7): forward primer, GTGCATGAAGCTCAACTCAACTATT (SEQ ID NO:8): reverse primer. GCCGCATTGGCATTCATGTT (SEQ ID NO:6).

Electrophoretic mobility shift assay. The nuclear proteins are extracted by a micro extraction method described by Osborn (1989) and minor modifications described by (Shi et al., 2003). Double-stranded Transcription factor binding oligonucleotides from Promega (Madison, Wis.) are used to determine DNA binding in nuclear proteins. The oligonucleotide is labeled with [γ-³²P] ATP and the EMSA reactions carried out in 30 μl of EMSA binding buffer (10 mM Tris-HCl [pH 7.5], 40 mM NaCl, 1 mM EDTA, 1 mM DTT) with 10 μg of nuclear extract, 0.5 ng of ³²P-labeled oligo, and 3 μg of poly (dI-dC/dI-dC). After incubation at 25° C. for 20 min, the reaction is stopped and products electrophoresed on non-denaturing 4% polyacrilamide gel in 0.5× Tris-borate EDTA at 160V. The specificity of bands is confirmed by competition with a 100-fold excess of unlabeled consensus oligonucleotide.

Supershift assays. Nuclear extracts prepared from cells in EMSA binding buffer, as described above, will be incubated with 2 to 4 μl of appropriate Transcruz (Santa Cruz Biotechnology, Santa Cruz, Calif.) supershift antibodies at 25° C. for 30 min before they are incubated with the [³²P]ATP labeled DNA protein EMSA binding buffer (Shi et al., 2003). Samples are electrophoresed for 90 min at 150V, as described above.

Transient cell transfections and luciferase activity. FuGENE 6 (Roche, Mannheim, Germany) is used to transfect luciferase-expressing pGL2 constructs in HCCSMC. The cells in passage 4 are seeded into 12-well plates (Costar, Acton, Mass.) and grown to 70% to 80% confluency. Transfections are carried out by incubation of cells in each well with 1.5 μl of FuGENE 6, 0.5 μg of pGL2 construct, and 0.125 μg of pSEAP2 Control Vector (BD Biosciences Clontech, Palo Alto, Calif.) for 24 h as per manufacturer's instructions. The pSEAP2 Control Vector is co-transfected for normalization of data. The transfected cells were treated with stimulus or medium control and harvested after 24 hours. Twenty μl of the supernatant of the cell lysates are taken for the measurement of luciferase activity by a luminometer (Promega, Madison, Wis.). The SEAP activity is measured with a Clontech SEAP detection kit by following the manufacturer's instructions.

Oligonucleotide agarose conjugate pull down assay. DNA affinity purification is performed as described previously (Al-Shami et al., 1998; Miura et al., 2001), with modifications. The oligonucleotides containing NF-κB-like sequences in hα1c1b promoter with 3′-terminal biotinylation and its complementary strand are synthesized by BioSource International, Camarelo, Calif. After annealing of the two single strand oligonucleotides, the double-stranded oligonucleotide was incubated with streptavidin-conjugated agarose beads (Pierce, Rockford, Ill.) for 1 h at 4° C. and washed twice with cell lysis buffer (CLB) (20 mM Tris-HCl (pH 7.5), 2 mM EDTA, 2 mM EGTA, 100 μg/ml aprotinin, 10 mM benzamidine, 5 mM DTT, 1 mM PMSF, 100 μg/ml leupeptin, 50 mM NaF, 5 mM Na₄P₂O₇, 1 mM Na₃VO₄, and 1% Nonidet P-40). Nuclear extract (20 μg) suspended in 300 μl of CLB was precleared with agarose beads for 1 h at 4° C. to remove any nonspecific binding to the beads. The lysates were then incubated with streptavidin-conjugated beads for 1 h at 4° C. The beads were washed three times with CLB buffer and the affinity-adsorbed protein was eluted by boiling in Laemmli buffer for 4 min at 95° C. and subjected to Western blotting.

Promoter constructs, 5′-deletions, and site-directed mutagenesis. The promoter constructs of hα1c1b are prepared by PCR amplification of human genomic DNA (Roche, Indianapolis, Ind.) and digested partially with Hind III (Promega, Madison, Wis.) using the FailSafe PCR kit (Epicentre, Madison, Wis.). The PCR product is subcloned into pDrive Cloning Vector with the PCR Cloning^{plus} kit (Qiagen, Valencia, Calif.) and SURE 2 supercompetent cells (Stratagene, La Jolla, Calif.) and subsequently transferred to the luciferase expression plasmid pGL2-Basic Vector (Promega, Madison, Wis.). The deletion constructs are made using the full length promoter construct as a template in PCR reactions. The same methods and kits are used to subclone the sequenced PCR products into the pDrive and pGL2 vector. The site-directed mutagenesis is carried out using QuikChange® II XL Site-Directed Mutagenesis Kit from Stratagene (La Jolla, Calif.), as per manufacturer's instructions.

Muscle bath experiments. Pure circular muscle strips (2 mm×10 mm) are prepared from resected human colon segments. The strips are mounted in 5 ml baths filled with carbonated Krebs solution (in mM: 118 NaCl, 4.7 KCl, 2.5 CaCl₂, 1 NaH₂PO₄, 1-2 MgCl₂, 11 D-glucose, and 25 NaHCO₃) at 37° C. (Siebenlist et al., 1994; Barnes and Karin, 1997). The muscle strips are equilibrated for 1 h at 1 g tension and then stretched until maximal response is obtained to 10⁻⁴ m ACh. The bathing solution is replaced every 15 min. Contractions are measured by force transducers and analyzed by DATAQ (Akron, Ohio) software.

Vertebrate Animals. Sprague-Dawley rats were used. For tissue harvesting, the rats were anesthetized with 5% isoflurame in 100% oxygen for 5 to 7 minutes followed by i.p. injection of 50 mg/kg ketamine.

Osmotic pumps were implanted under anesthesia as discussed above. Lateral surface of left back were shaven and cleaned with betadine. A 2 cm incision was made in the skin and subcutaneous facia and muscle separated. The micro-osmotic pump were implanted intramuscularly and the incision was closed.

Colonic inflammation was induced by intraluminal injection of 30 mg/kg or 130-mg/kg trinitrobenzene sulfonic acid (TNBS) under anesthesia, as described above.

Hormone Peptides. VIP and PACAP used in the experiments were obtained from Bachem Biosource, King of Prussia, Pa. Unless otherwise noted, 38-(PACAP38) was used.

Ribonuclease protection assay (RPA): Ten micrograms of total RNA extracted from HCCSMC with the RNeasy Mini isolation kit (QIAGEN Inc, Valencia, Calif.) were subjected to RPA utilizing RPA III MAXIscript T7 kits from Ambion, Inc. (Austin, Tex.) according to the manufacturer's instructions. Two probes were utilized to detect the presence of transcripts containing exon 1a or exon 1b as described by Saada et al. Briefly, one probe was generated by RT-PCR from human heart RNA using a sense primer GCGATGCGATACGGCCATGTC in the 5 untranslated region (utr) of exon 1a and antisense primer TGGAGCTGACTGTGGAGATG in the second exon. This probe was subcloned with a TA cloning kit into pCR 2.1 (Invitrogen, Carlsbad, Calif.), sequenced, and digested with DraI (shortening it by 112 nucleotides), and a riboprobe was generated using the T7 promoter (13). In RPA, this probe generates a longer protected fragment of 350 bp for hα_{1C}1a transcripts containing exon 1a and a shorter 212 bp protected fragment for transcripts not containing exon 1a.

The second probe was generated by RT-PCR from bladder RNA using a sense primer CGTGGCTGCTCCTCCTATTA in the 5 utr of exon 1b and antisense primer TGGAGCTGACTGTGGAGATG in exon 2. This probe was subcloned also into pCR 2.1 and digested with HindIII. This probe was susceptible to cleavage near one end and so it was shortened by digestion with SmaI near the 3′ end of the insert and EcoRV on the vector. The fragment which included the rest of the insert and the vector was gel purified and religated, and the vector was digested with HindIII to access the T7 promoter. This probe generates a larger protected segment of 173 bp for hα_{1C}1b transcripts containing exon 1b and a shorter 92 bp protected segment not containing exon 1b. Both probe sequences were verified by DNA sequencing.

Nuclear run-on assay: Nuclei were isolated from cultured HCCSMC according to the method of Dignam. Briefly, cells (2×10⁷ cells/assay) were washed twice with ice-cold phosphate-buffered saline (PBS), scraped and collected in a 15 mL centrifuge tube by centrifugation at 500 g for 5 min at 4° C. Subsequent steps were performed at 4° C. The cells were resuspended in 4 mL of lysis buffer (in mM: 10 Tris-HCl [pH 7.4], 10 NaCl, 3 MgCl₂ and 0.5% Nonidet P-40) and allowed to stand on ice for 5 min. and then centrifuged at 500 g at 4° C. for 5 min. Nuclei were resuspended in 200 μL of glycerol storage buffer (in mM: 10 Tris-HCl [pH 8.3], 5 MgCl₂, 0.1 EDTA; 40% (v/v) glycerol) and frozen in liquid N2. In vitro transcription and isolation of the resulting nuclear RNA were performed as described by Ikeda et al. Two-hundred μL of frozen nuclei were thawed and mixed with 200 μL of 2× reaction buffer (in mM: 10 Tris-HCl [pH 8.0], 5 MgCl₂, 300 KCl, 10 dithiothreitol, 20 creatine phosphate and 400 units/mL placental ribonuclease inhibitor, 200 μg/mL creatine phosphokinase, 1 mM concentration each of ATP, CTP, and GTP and 100 μCi of [α-³²P]UTP (3000 Ci/μmol, Amersham, Piscataway, N.J.). Samples were incubated at 30° C. for 30 min while shaking and for 5 min in the presence of 20 units of DNase I. After the addition of proteinase K (150 μg/mL) and SDS (0.5% final concentration), incubation was continued at 37° C. for 30 min. Extracted RNA was resuspended in TES buffer (in mM: 10 TES at pH 7.4, 10 EDTA; and 0.2% SDS) at 5×10⁶ cpm/mL. Linearized plasmids (α_{1C} cDNA was a generous gift from Dr. Nikolai Soldatov, National Institute of Aging, NIH; IL-8 cDNA, a gift from Antonella Cassola University of Texas Medical Branch; and β-actin cDNA, HHCJ95 from American Type Culture Collection, Manassa, Va.) containing the target cDNAs (15 μg) were immobilized onto a nylon Duralon-UV membrane (Stratagene, La Jolla, Calif.) using a Bio-Dot SF microfiltration apparatus (Bio-Rad, Hercules, Va.). The filters were prehybridized overnight at 42° C. with hybridization buffer containing in mM: 20 PIPES, at pH 6.4, 2 EDTA, 800 NaCl; and 50% formamide, 0.2% SDS, 1×Denhardt's solution (0.02% Ficoll, 0.02% BSA, 0.02% polyvinylpyrrolidone), 200 μg/mL E. coli tRNA (RNase-free). Hybridization was performed at 42° C. for 48 h in the same solution supplemented with 15×10⁶ total cpm of labeled RNA. The filters were washed twice in 2×SSC, 0.5% SDS at 42° C. for 30 min, twice in 0.3×SSC, 0.5% SDS, at 42° C. for 30 min and then incubated with 10 μg/mL RNase A in 2×SSC at 37° C. for 30 min. Further washings were done in 2×SSC at 37° C. for 30 min and then in 0.3×SSC at 37° C. for 30 min. The filters were exposed on autoradiography with Hyperfilm-MP and intensifying screens at −80° C. The amount of α_{1C} mRNA was standardized by comparison with β-actin mRNA.

Transfection of phosphorothioated sense and antisense oligonucleotides in HCCSMC and colonic circular muscle strips: Phosphorothioated sense and anti-sense oligonucleotides were synthesized commercially and cartridge purified by Biosource International (Foster City, Calif.). The sequences of the human p65 sense and antisense, and p50 sense and antisense oligonucleotides were: 5′-GCCATGGACGAACTGTTCCCC-3′; 5′-GGGGAACAGTTCGTCCATGGC-3′; 5′-AGAATGGCAGAAGATGATCCA-3′; and 5′-TGGATCATCTTCTGCCATTCT-3′ respectively.

Cells (5×10⁴ in 1 mL medium) were seeded into each well of a 12-well culture plate one day prior to transfection with sense and anti-sense oligonucleotides. The cells were washed with PBS and treated with 4 μM oligonucleotides in the medium in the presence of 1.5 μL FuGENE 6 (Roche, Mannheim, Germany) for 24 h. Then TNFα or control medium was added to the cells for 24 h before harvesting them.

The protocol for transfection of sense and antisense oligonucleotides in freshly obtained muscle strips was similar to that in HCCSMC. The circular muscle layer was isolated from the fall thickness human colon, and sliced into 2 mm×10 mm muscle strips. The strips were incubated in RPMI 1640 without serum. The sense and antisense oligonucleotides (10 μM) were added into the medium for 24 h before incubation with 20 ng/mL TNFα for 24 h.

Rapid Amplification of cDNA Ends (RACE): The transcription start site of hα_{1C}1b transcript was determined with the FirstChoice RACE-Ready cDNA for human colon from Ambion (Austin, Tex.). Gene-specific primer was 5′-GTTTTCCTCTGGAATGTACA-3′. The PCR products were subcloned into pCR2.1-TOPO (Invitrogen, Carlsbad, Calif.), and sequenced. The transcription start site in HCCSMC was confirmed by PCR with cDNAs made from the RNA extracted from HCCSMC.

Cytosolic free Ca²⁺ measurement: Cytosolic calcium level ([Ca²⁺]i) was measured in a wide-field imaging system at the UTMB core Optical Imaging Laboratory using the Ca²⁺-sensitive fluorescent dye fura 2-AM (Molecular Probes, Eugene, Oreg.). HCCSMC cultured on cover slips were incubated with 1 μM fura 2-AM for 45 min at 37° C. in a modified HEPES buffer (pH 7.4) containing (in mM): 10 HEPES, 125 NaCl, 5 KCl, 1 CaCl₂, 0.5 MgSO₄, 5 glucose. Cover slips with cells were washed with fresh medium and mounted on the stage of a Nikon TE200 inverted microscope (Nikon Instruments, Lewisville, Tex.) equipped with a CoolSNAP HQ cooled-CCD monochrome digital camera (Roper Scientific, Tucson Ariz.). Cells were examined under a super-fluor 20×0.75 NA objective during exposure to alternating 340 and 380 nm excitation light (DG4 illuminator system, Sutter Instrument, Novato, Calif.), and the intensity of light emission at 510 nm was measured. The light intensities and their ratio, F₃₄₀/F₃₈₀, which reflects changes in Ca²⁺ concentration, were followed simultaneously in several single cells in the field. The acquisition process was controlled by the Metafluor image acquisition software (Universal Imaging Corp. Downingtown, Pa.).

### Example 2

### VIP and PACAP Induce Expression of α1C

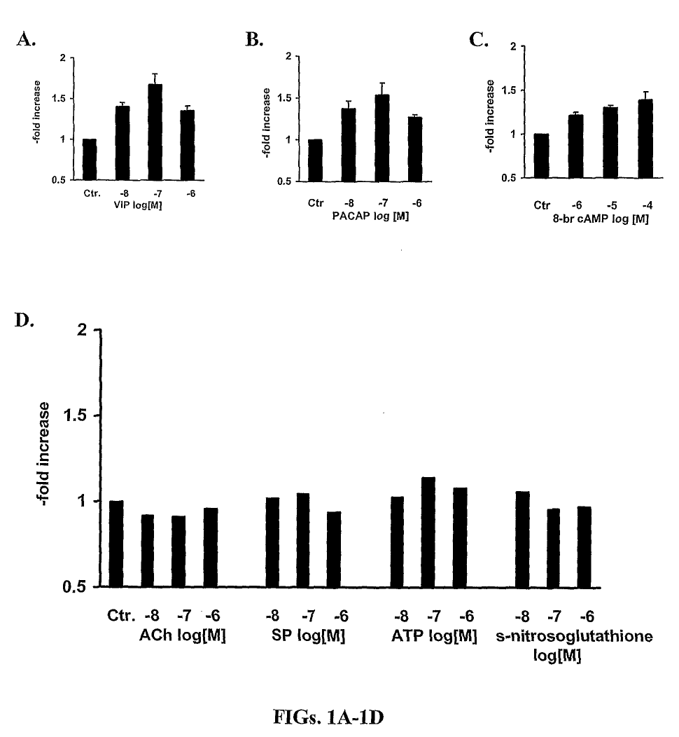

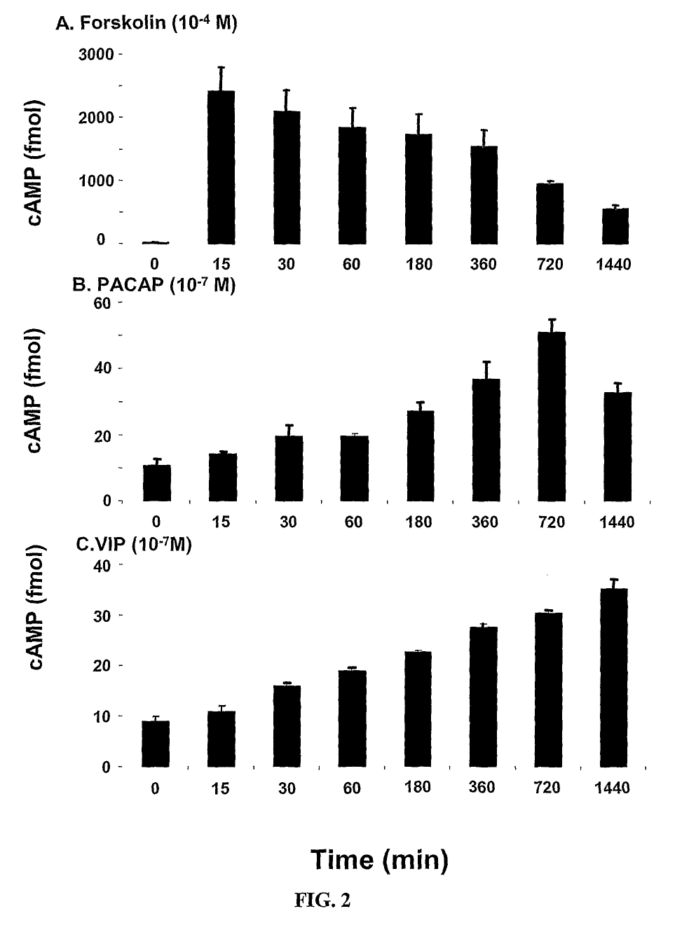

The potential of all major neurotransmitters of enteric motor neurons (ACh, SP, ATP, NO, 8-bromo cAMP, VIP and PACAP) was tested for induction of the mRNA expression of α_{1C}. VIP and PACAP induced expression (FIG. 1). FIG. 1 indicates that the expression of the α_{1C} subunit of L-type Ca²⁺ channels is maximal between the concentrations of 10⁻⁸ M to 10⁻⁷ M. These studies showed that VIP increased α_{1C} mRNA by about 50% at 6 h and α_{1C} protein by about 100% in adult human colonic circular smooth muscle cells. It is noteworthy that these increases are smaller than those seen for inducible proteins, such as inflammatory mediators (Silva et al., 1998). This may be for two reasons, one that L-type calcium channel protein is expressed constitutively and, therefore, there is already a large denominator for determining -fold increase in contrast to the very small basal amounts of inducible proteins that provide a small denominator. Also, the increase of calcium channels may be restrained by endogenous factors to limit excessive Ca²⁺ influx which can be detrimental to the cells. Nevertheless the increase of L-type calcium channels in HCCSMC significantly enhances the contractile response to ACh. In additional experiments, norepinephrine, which also activates adenylyl cyclase, induced the expression of α_{1C} mRNA.

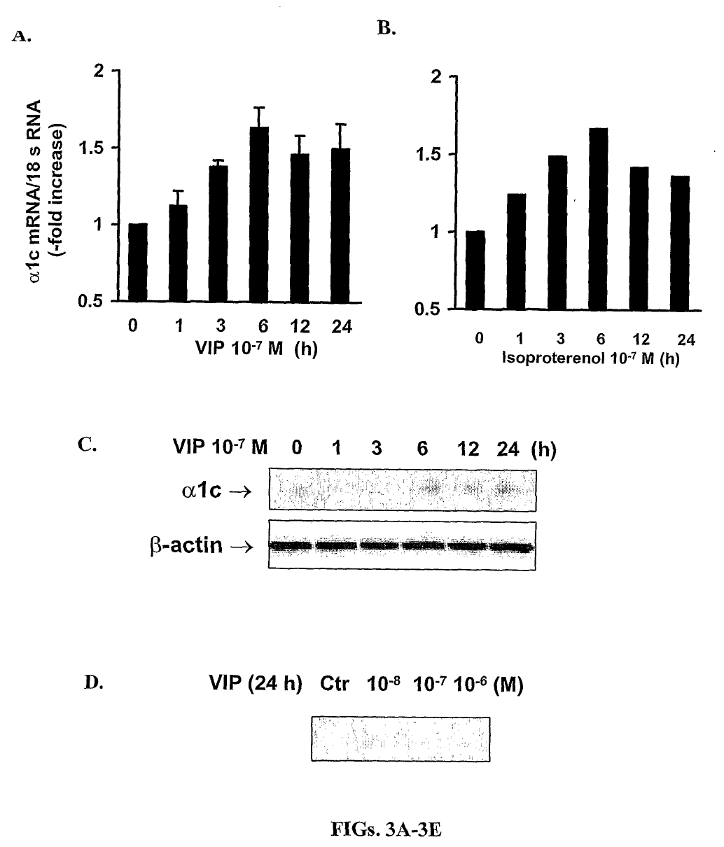

Moreover, the intercellular cAMP levels were elevated throughout the 24 h period of VIP treatment (FIG. 2). These data would suggest that there may not be any refractory period for the generation of cAMP in HCCSMC (Montminy, 1997). Therefore, cAMP/PKA signaling pathway would be activated continuously in these cells to induce α_{1C} gene for at least 24 h, as seen in the data for VIP in FIG. 3.

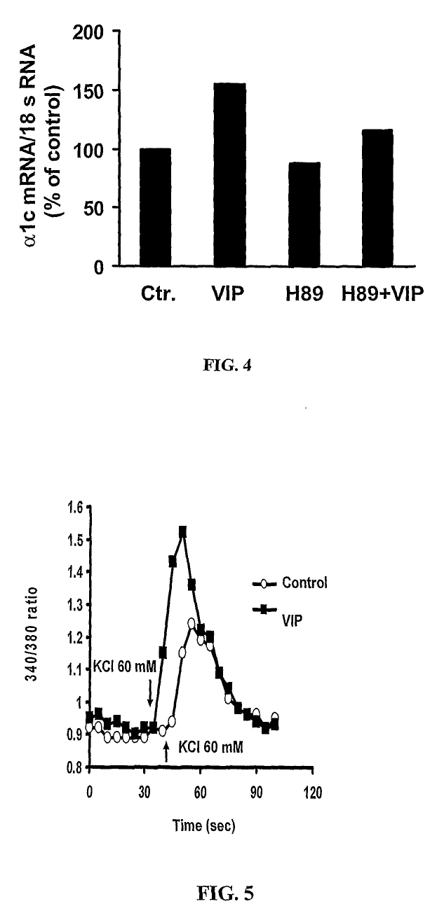

According to data shown in FIG. 4, it is expected that both VIP and PACAP will enhance α_{1C} mRNA and protein expression through cAMP-dependent and cAMP independent pathways, suggesting that multiple signaling pathways may be involved in the overall induction of α_{1C} gene by VIP/PACAP. G_{αs} and Gα_{q/ll} are both expected to mediate the responses to VIP and PACAP, but in different proportions. It is also expected that Ca²⁺ influx induced by KCl and BAY K8644 would be potentiated in cells treated with VIP/PACAP for 24 h in agreement with the increased expression of the pore-forming α_{1C} subunit of L-type calcium channels (FIG. 5).

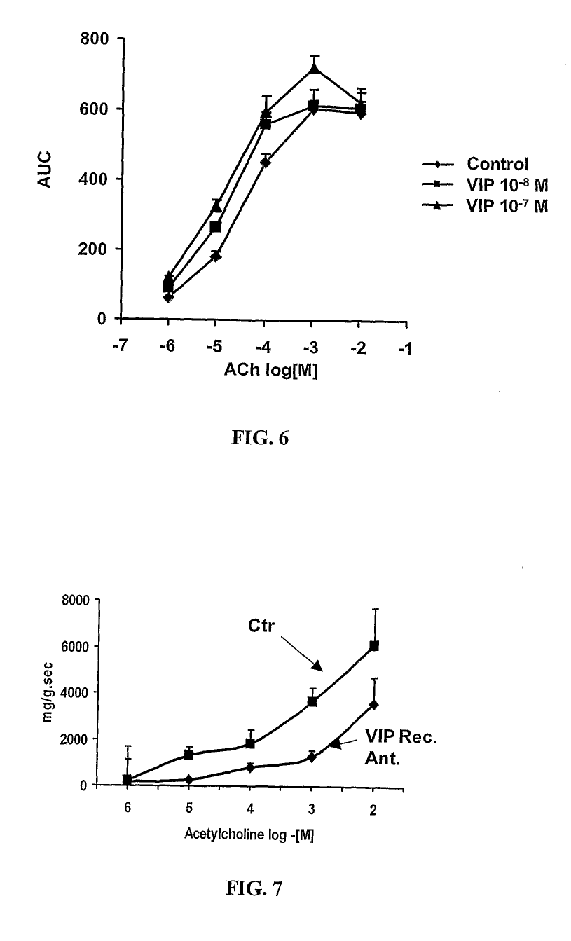

Incubation of circular muscle strips with VIP for 24 h enhanced the contractile response to ACh and KCl in organ baths (FIG. 6).

Administration of VIP/PACAP receptor antagonists in intact conscious rats by osmotic pump for 3 days reduced the contractile response of their colonic circular muscle strips to ACh (FIG. 7) and α_{1C} expression.

Overall, these data show systemic administration of very low doses of VIP/PACAP will enhance the expression of L-type calcium channels and hence increase Ca²⁺ influx to potentiate smooth muscle contractions.

### Example 3

### Regulation of α_{1C} Subunit Promoter

Data using promoter sequence analyzer programs (MatInspector, Genomatrix, Munich, Germany, and Transcription element search software (TESS)) have identified two potential CRE elements on human α1C1b promoter at −563/−556 and −176/−169 from the transcription start site. These sites are separated by 387 bases. The 5′-CRE (CRE1), TGACGTCA, is a consensus sequence, whereas the 3′-CRE (CRE2), TGACAGCA, is a variant sequence with 80% homology to the consensus sequence. Using immunofluorescence imaging CREB was shown to be a resident protein in the nucleus of HCCSMC and it is phosphorylated upon exposure to VIP.

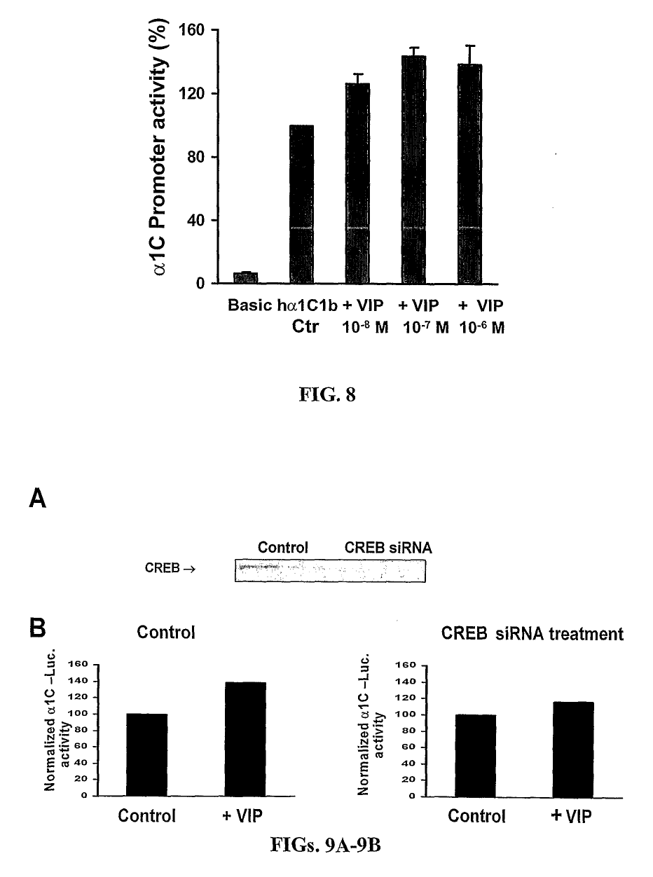

Data using a wild type α1C1b promoter subcloned upstream of firefly luciferase reporter gene derived from a PSV40 vector (Pazdrak et al., 2004; Weih et al., 1996) established that the activity of this construct is enhanced in a concentration-dependent manner by treatment with VIP (FIG. 8).

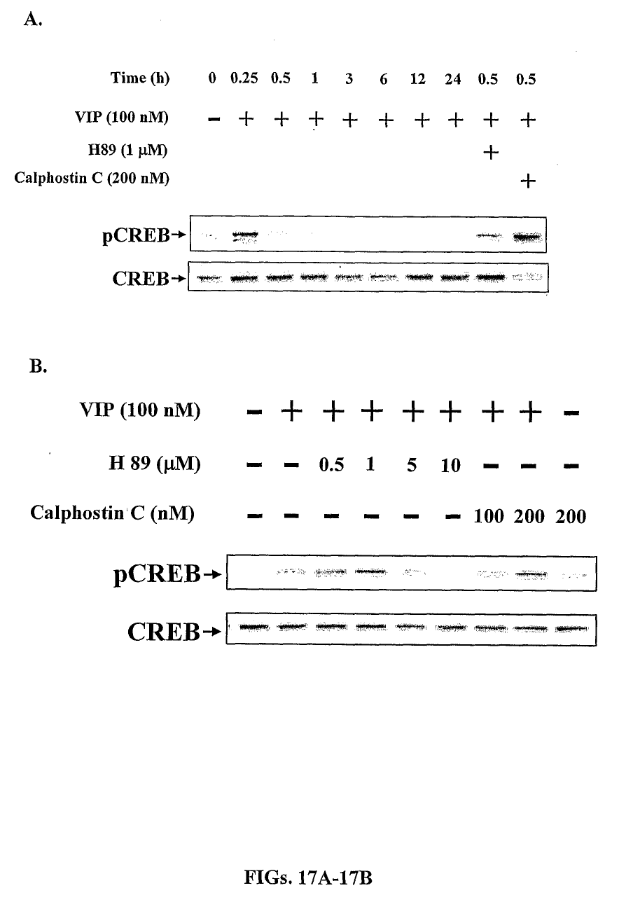

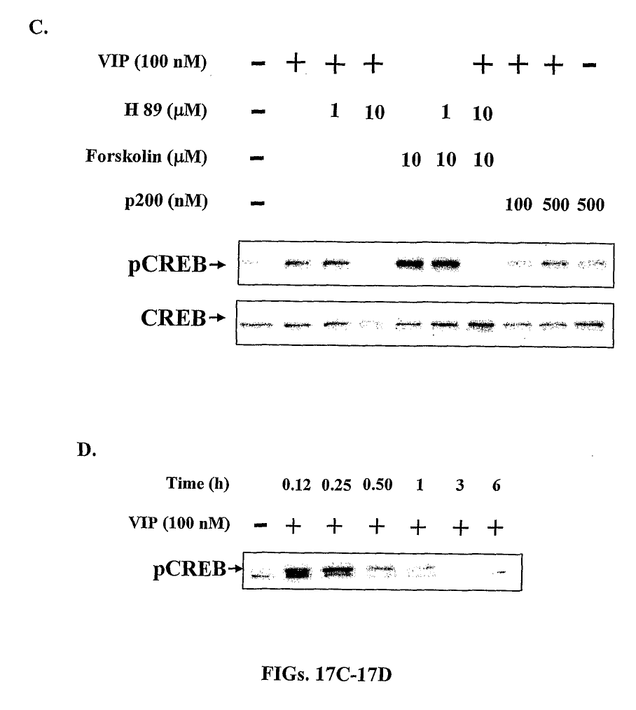

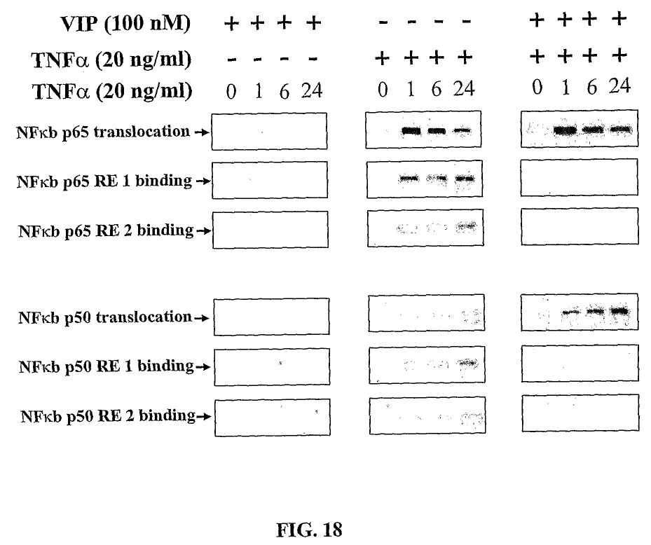

VIP treatment led to only transient phosphorylation of CREB (100 nm CREB maximally phosphorylated between 15 and 30 min). (FIG. 17D). CREB phosphorylation by VIP lasted about one hour. The confirmation of this finding in further experiments would suggest that CREB phosphorylation may be required only to initiate the transcription of α_{1C} gene but it may not be essential to maintain it. As seen in data of FIG. 3, the transcription of α_{1C} mRNA and its translation continue for at least 24 h after VIP treatment.

Data in FIG. 9A-9B was generated using siRNA. This confirmed that CREB is essential for activation of promoter α1C1b. Transient transfection of the cells with siRNA of CREB (SMARTpool CREB siRNA from Dharmacon RNA Technologies, Lafayette, Colo.) almost completely blocked the α1C1b promoter reporter activity. This suggests that the long-term transcription of α_{1C} may be maintained by a co-transcription factor.

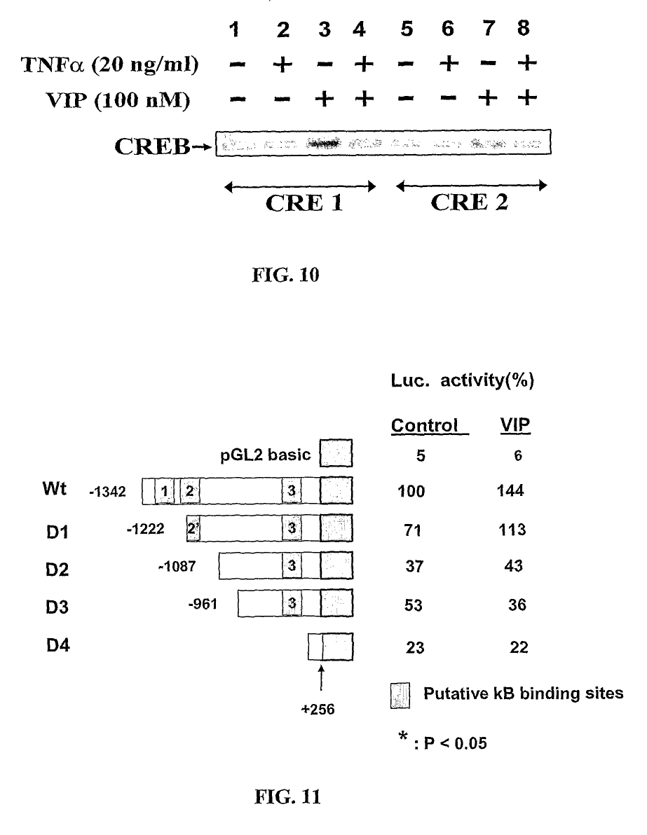

Agarose-nucleotide pull-down assays indicated that CREB will bind to both CRE1 and CRE2 (FIG. 10). It appears CREB binds to the CRE2 motif with a lower affinity than to the CRE1 motif because CRE2 is a variant sequence and CRE1 is a consensus sequence. This difference would suggest a smaller cis-activating potential of CRE2 motif than that of CRE1 motif in deletion and mutation experiments. Moreover, it appears that more CREB binds at both CRE1 and CRE2 in the presence of VIP. This effect is abrogated by TNFα. (FIG. 10). According to the data for two 5′-deletions (FIG. 11), one or both CRE motifs in α1C1b promoter have cis-potential.

Other data showed that the concurrent binding of p50/p65 NF-κB subunits to two κB motifs was necessary for the repression of α_{1C} gene. However, in that case the two κB motifs were separated by only 16 bases in contrast to 387 bases for CRE motifs.

The MAPK signaling pathway is one of the most conserved in all eukaryotes (English et al., 1999; Houslay and Kolch, 2000) and it has the same versatility and importance as the cAMP/PKA signaling pathway. In many cells, the interactions between the cAMP/PITA and MAPK signaling pathways are critically important in regulating cellular functions (English et al., 1999; Houslay and Kolch, 2000). Experiments indicated that VIP time-dependently dephosphorylated the three MAP kinases: ERK ½, p38 and JNK/SAPK.

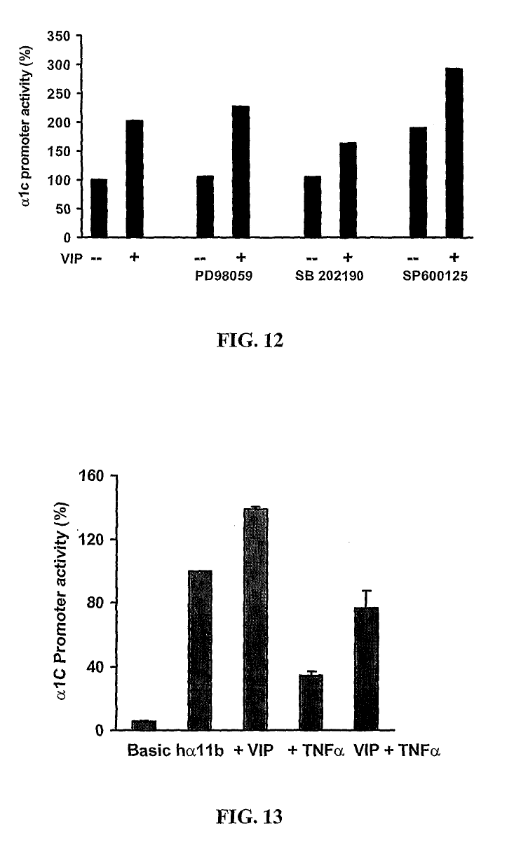

According to the data in FIG. 12, the antagonist of ERK ½ (PD98059) may have little effect on constitutive α1C1b promoter activity or on its enhancement by VIP. p38 also may have little effect on constitutive promoter activity, but it may somewhat blunt the enhancement by VIP (SB202190-p38 inhibitor). Thus, it is likely that these kinases may not be involved in the enhancement of α_{1C} gene by VIP/PACAP, although, this remains to be confirmed in further studies. Conversely, it is suspect that constitutive phosphorylation of JNK/SAPK represses α_{1C} transcription. The inhibition of this kinase by SP600125 is, therefore expected to increase promoter activity (FIG. 12) and α_{1C} protein. A part of the effect of VIP/PACAP on the up-regulation of α_{1C} gene may be mediated, therefore, by dephosphorylation of JNK/SAPK (FIG. 12). The effects of JNK/SAPK inhibitor SP600125 and VIP are expected to be additive. Thus, while VIP dephosphorylates all three MAPKs, only JNK/SAPK may be involved in the up-regulation of α_{1C} gene.

### Example 4

### VIP/PACAP Effect on TNFα Treatment

The abundance of p65 subunit in the nucleus on treatment of HCCSMC did not seem to be different whether with TNFα alone or treatment with VIP and TNFα. TNFα enhanced the nuclear abundance and binding of p65 in a time-dependent manner. Accordingly, VIP/PACAP probably will not affect the phosphorylation and degradation of IκBs. Similar results are expected for p50. It is likely that one of the factors for inhibition of the binding of NK-κB subunits to α1C1b promoter is the utilization of significant quantities of CREB binding protein (CBP) that is required by numerous transcription factors to bind to their promoters (Parry and Mackman, 1997; Bito and Takemoto-Kimura, 2003). CBP is present in limited quantities in cells and is often a rate limiting factor in transcription (Parry and Mackman, 1997; Bito and Takemoto-Kimura, 2003). Other factors that may mediate the above effects of VIP/PACAP may be the interactions among cAMP/PKA and MAPK signaling pathways. Consistent with the reduction of NF-κB binding to repressive cis-motifs on α1C1b promoter, TNFα alone reduced luciferase activity of the α1C1b promoter driven luciferase gene and VIP/PACAP significantly blocked this reduction (FIG. 13).

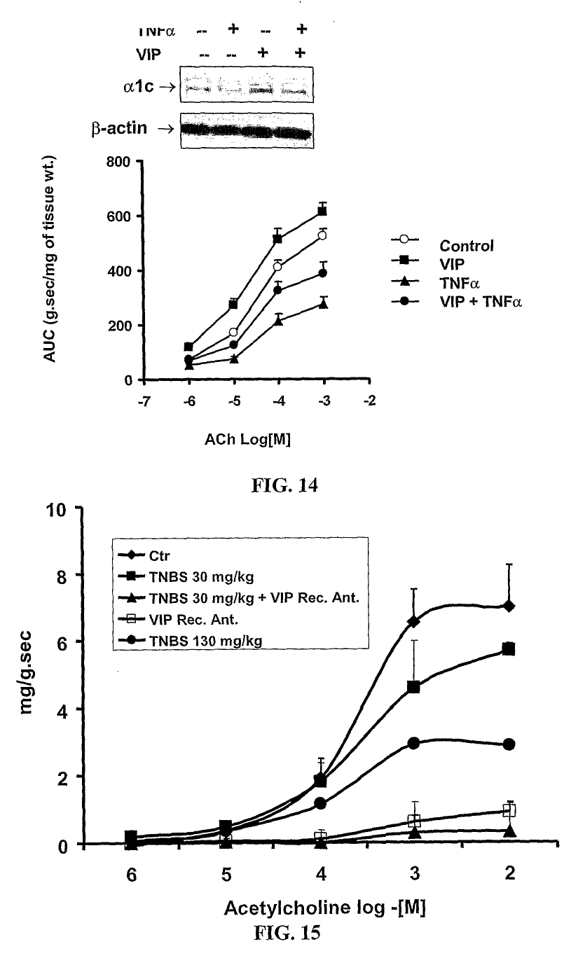

Muscle bath experiments showed that TNFα-treatment of muscle strips alone suppresses their contractile response to ACh, but pre-treatment with VIP/PACAP significantly reduces the suppression (FIG. 14). At the same time the suppression of α_{1C} protein in muscle strip tissue was minimized by VIP/PACAP (top, FIG. 14).

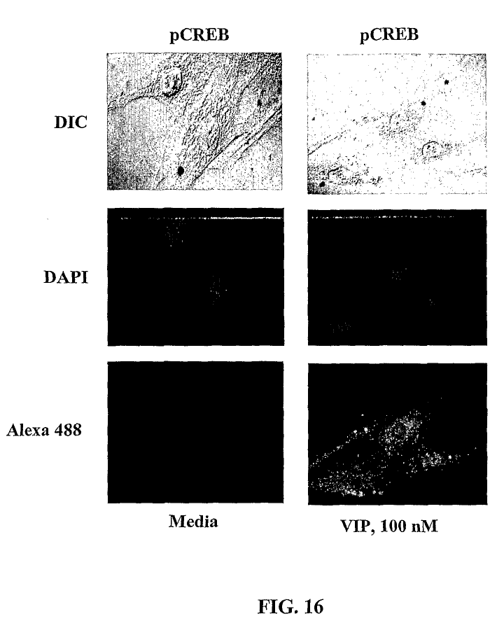

The receptor antagonists of VIP/PACAP are expected to enhance the suppressive effects of TNFα in muscle strips. It is expected that in the intact rat model VIP/PACAP-treatment through osmotic pumps would partially inhibit the suppression of contractility due to TNBS-induced inflammation in all parts of the colon. On the contrary, co-treatment with VIP/PACAP receptor antagonist through osmotic pumps enhanced the suppression of contractility so that a significant reduction was seen with the sub-threshold dose of TNBS that produced minimal effects by itself (FIG. 15). Observations indicated that the rats treated with VIP receptor antagonist (p-chloro-D-Phe⁶, Leu¹⁷)-VIP followed by the sub-threshold dose of TNBS (30 mg/kg) exhibited greater diarrhea and weight loss than those treated with the regular dose of TNBS (130 mg/kg). The rats treated with the receptor antagonist alone did not develop diarrhea.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,962,436
U.S. Pat. No. 4,215,051
U.S. Pat. No. 4,503,031
U.S. Pat. No. 4,554,101
U.S. Pat. No. 4,605,641
U.S. Pat. No. 4,624,665
U.S. Pat. No. 4,781,924
U.S. Pat. No. 4,808,416
U.S. Pat. No. 4,866,039
U.S. Pat. No. 4,889,721
U.S. Pat. No. 4,908,027
U.S. Pat. No. 4,939,224
U.S. Pat. No. 4,942,037
U.S. Pat. No. 5,028,592
U.S. Pat. No. 5,055,307
U.S. Pat. No. 5,399,363
U.S. Pat. No. 5,412,087
U.S. Pat. No. 5,436,009
U.S. Pat. No. 5,445,934
U.S. Pat. No. 5,466,468
U.S. Pat. No. 5,500,221
U.S. Pat. No. 5,543,158
U.S. Pat. No. 5,641,515
U.S. Pat. No. 5,681,816
U.S. Pat. No. 5,726,154
U.S. Pat. No. 5,744,305
U.S. Pat. No. 5,846,225
U.S. Pat. No. 5,846,233
U.S. Pat. No. 5,863,555
U.S. Pat. No. 5,879,703
U.S. Pat. No. 5,990,179
U.S. Pat. No. 6,007,837
U.S. Pat. No. 6,045,824
U.S. Pat. No. 6,074,673
U.S. Pat. No. 6,136,320
U.S. Pat. No. 6,284,262
U.S. Pat. No. 6,309,823
U.S. Pat. No. 6,316,193
U.S. Pat. No. 6,322,971
U.S. Pat. No. 6,324,479
U.S. Pat. No. 6,329,140
U.S. Pat. No. 6,329,209
U.S. Pat. No. 6,488,953
U.S. Pat. No. 6,521,250
U.S. Pat. No. 6,737,045
U.S. Pat. No. 6,794,357
U.S. Pat. No. 6,797,258
U.S. Patent Appln. 20030104468
Abad et al., Gastroenterology, 124(4):961-971, 2003.
Abernethy and Soldatov, J. Pharmacol. Exp. Ther., 300(3):724-728, 2002.
Akbarali et al., Biochem. Biophys. Res. Commun., 275(2):637-642, 2000.
Al-Shami et al., J. Biol. Chem., 273(2):1058-1063, 1998.
Angel and Karin, Biochim. Biophys. Acta, 1072(2-3):129-157, 1991.
Anlese et al., Scand. J. Gastroenterol., 32:1107-1117, 1997.
Ausubel et al., In: Current Protocols in Molecular Biology, John, Wiley & Sons, Inc, New York, 1996.
Bampton et al., Am. J. Gastroenterol., 95:1027-1035, 2000.
Bannister and Kouzarides, Nature, 384(6610):641-643, 1996.
Barany and Merrifield, In: The Peptides, Gross and Meienhofer (Eds.), Academic Press, NY, 1-284, 1979.
Barnes and Karin, N. Engl. J. Med., 336(15):1066-1071, 1997.
Bassotti et al., Am. J. Gastroenterol., 94:1760-1770, 1999.
Bassotti et al., J. Int. J. Colorectal Dis., 19:493-497, 2004.
Bayliss and Starling, Am. J. Physiol., 24:99-143, 1899.
Beyerman et al., Life Sci., 29(9):895-902, 1981.
Biancani et al., Am. J. Physiol., 266:G767-774, 1994.
Bito and Takemoto-Kimura, Cell Calcium., 34(4-5):425-430, 2003.
Bittner et al., Methods in Enzymol, 153:516-544, 1987.
Bodansky et al., J. Med. Chem., 21(11):1171-1173, 1978.
Bornstein et al., Neurogastroenterol. Motil., 16(1):34-38, 2004.
Brinkmann and Pastan, Biochim. Biophys. Acta, 1198(1):27-45, 1994.
Bueno et al., Gut., 21:480-485, 1980.
Burgering et al., EMBO J., 12(11):4211-4220, 1993.
Cambi et al., J. Neurochem., 53(5):1656-1659, 1989.
Canfield et al., Methods Eiizymol., 189:418-422, 1990.
Capaldi et al., Biochem. Biophys. Res. Comm., 74(2):425-433, 1977.
Caruthers et al., Nucleic Acids Symp. Ser., (7):215-223, 1980.
Catalysis in Micellar and Macromolecular Systems, Fendler and Fendler (Eds.), NY, Academic Press, 1975.
Catterwall, Science, 242(4875):50-61, 1988.
Chen et al., Cancer Res., 59(1):213-218, 1999.
Chen et al., Neurogastroenterol. Motil., 17:878-882, 2005.
Chey et al., Am. J. Gastroenterol., 96:1499-1506, 2001.
Chow and Kempe, Nucleic Acids Res., 9(12):2807-2817, 1981.
Cicalese et al., Transplant Proc., 28(5):2459, 1996.
Clemens et al., Am. J. Gastroenterol., 98:1838-1843, 2003.
Coffin et al., Am. J. Physiol., 267:G569-574, 1994.
Colbere-Garapin et al., J. Mol. Biol., 150:1-14, 1981.
Colloidal Surfactants, Shinoda et al. (Eds.), NY, Academic Press, 1963.
Cook et al., Am. J. Physiol. Gastrointest Liver Physiol., 278:G329-341, 2000.
Couvineau et al., Biochem. Biophys. Res. Commum., 121(2):493-498, 1984.
Cowles and Sarna, Am. J. Physiol., 259:G420-429, 1990.
Crea and Horn, Nucleic Acids Res., 8(10):2331-2348, 1980.
Creighton In: Proteins, Structures and Molecular Principles, W.H. Freeman and Co., NY, 34-49, 1983b.
Creighton, In: Proteins Structures And Molecular Principles, W.H. Freeman and Co., NY, 50-60, 1983a.
Dai et al., Biochem. Biophys. Res. Commun., 296(2):429-433, 2002.
Delgado et al., J. Biol. Chem., 273(47):31427-31436, 1998.
DeRisi et al., Nature Genetics, 14:457-460, 1996.
Efstratiadis et al., Proc. Natl. Acad. Sci. USA, 73(7):2289-2293, 1976.
Ekblad, Regul. Pept., 85(2-3):87-92, 1999.
El-Gorab et al., Biochim. Biophys. Acta, 306(1):58-66, 1973.
Ellis et al., Science, 241(4873):1661-1664, 1988.
English et al., Exp. Cell Res., 253(1):255-270, 1999.
EP 0 072 251
Eskandari et al., Am. J. Physiol., 273(3 Pt 1):G727-734, 1997.
Eskandari et al., Transplant Proc., 30(6):2670, 1998.
Fan et al., Biochim. Biophys. Acta, 1577(3):401-411, 2002.
Ford et al., Gut., 37:264-269, 1995.
Forrsmann et al. (eds.), Ann. N.Y. Acad. Sci. 865, 1998.
Foulkes et al., Proc. Natl. Acad. Sci. USA, 88(12):5448-5452, 1991.
Frantzides et al., Gastroenterology, 92:594-603, 1987.
Freshney, In: Animal Cell Culture, A Practical Approach, 2^{nd} Ed., Oxford Press, UK, 1992.
Furness et al., Arch. Histol. Cytol., 53(2):129-140, 1990.
Galligan, Curr. Opin. Pharmacol., 2:623-629, 2002.
Gardner et al., Endocrinol. Jpn., 27(1):1-5, 1980.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh et al., Annu. Rev. Immunol., 16:225-260, 1998.
Gonzalez and Montminy, Cell, 59(4):675-680, 1989.
Gozes et al., Ann NY Acad Sci., 805:159-169, 1996.
Grider and Rivier, J. Pharmacol. Exp. Ther., 253(2):738-742, 1990.
Grider et al., Am. J. Physiol., 270:G778-782, 1996.
Glider et al., J. Auton. Nerv. Syst., 50(2):151-159, 1994.
Grider, Am. J. Physiol. Gastrointest. Liver Physiol., 284(5):G768-775, 2003.
Grider, J. Pharm. Exper. Therap., 307(2):460-467, 2003.
Gubler et al., Gene, 25:263-269, 1983.
Haba and Sarna, Am. J. Physiol., 264:G261-271, 1993.
Hacia et al., Nature Genet., 14:441-449, 1996.
Hai et al., Genes Dev., 3(12B):2083-2090, 1989.
Harmar et al., Endocrinology, 145(3):1203-1210, 2004.
Harmar et al., Pharmacol. Rev., 50(2):265-270, 1998.
Hartman and Mulligan, Proc. Natl. Acad. Sci. USA, 85:8047-8051, 1988.
Higuchi et al., Proc. Natl. Acad. Sci. USA, 73(9):3146-3150, 1976.
Hou et al., Am. J. Physiol., 256(6 Pt 1):G997-1004, 1989.
Houslay and Kolch, Mol. Pharmacol., 58(4):659-668, 2000.
Huang et al., Am. J. Physiol. Gastrointest Liver Physiol., 288:G23-31, 2005.
Jay et al., Science, 248(4954):490-492, 1990.
Johnson et al., In: Biotechnology And Pharmacy, Pezzuto et al. (Eds.), Chapman and Hall, NY, 1993.
Johnson et al., Surgery, 121:182-189, 1997.
Jouet and Sarna, J. Pharmacol. Exp. Ther., 279:207-213, 1996.
Jouet et al., Am. J. Physiol., 274:G459-464, 1998.
Kalff et al., J. Leukoc. Biol., 63(6):683-691, 1998.
Karaus and Sarna, Gastroenterology, 92:925-933, 1987.
Khokhlatchev et al., Cell, 93(4):605-615, 1998.
Kim et al., J. Biol. Chem., 265(20):11858-11863, 1990.
Ko et al., Plant Mol. Biol., 14(2):217-227, 1990.
Komatsu et al., Clin. Chem., 47(7):1297-1301, 2001.
Kwok et al., Nature, 370(6486):223-226, 1994.
Kyte and Doolittle, J. Mol. Biol., 157(1):105-132, 1982.
Lee and Masson, Biochim. Biophys. Acta. 1174(3):221-133, 1993.
Liu et al., Am. J. Gastroenterol., 100:792-796, 2005.
Liu et al., Gastroenterology, 120(2):480-489, 2001.
Liu et al., Gastroenterology, 120:480-489, 2001.
Lomax and Furness, Cell Tissue Res., 302(1):59-72, 2000.
Lomax et al., J. Auton. Nerv. Syst., 76:45-61, 1999.
Lomax et al., Neurogastroenterol. Motil., 17:4-15, 2005.
Lonze and Ginty, Neuron., 35(4):605-623, 2002.
Lowy et al., Cell, 22:817-823, 1980.
Lutz et al., FEBS Lett., 334(1):3-8, 1993.
Makhlouf and Murthy, Cell Signal, 9:269-276, 1997.
Maki et al., J. Clin. Invest., 97(3):656-663, 1996.
Maniatis et al., Cell, 8:163, 1976.
Maniatis, et al., Molecular Cloning, A Laboratory Manual, Cold Spring Harbor Press, Cold Spring Harbor, N.Y., 1990.
Matsufuji and Yokoyama, J. Smooth Muscle Res., 39:11-20, 2003.
Matsushima, Nippon Heikatsukin Gakkai Zasshi, 25:137-146, 1989.
McConalogue et al., Balliere's Clin. Endocrin. Metab. 8:51-76, 1994.
McConlogue, In: Current Communications in Molecular Biology, Cold Spring Harbor Laboratory, 1987.
Merrifield, Science, 232(4748):341-347, 1986.
Miampamba and Sharkey, Neurogastroenterol. Motil., 10(4):315-329, 1998.
Mikami et al., Nature, 340(6230):230-233, 1989.
Miura et al., J. Immunology, 167:2282-2291, 2001.
Modern Pharmaceutics, 1990
Montminy, Annu. Rev. Biochem., 66:807-822, 1997.
Montminy, Proc. Natl. Acad. Sci. USA, 83(18):6682-6686, 1986.
Morse and Sassone-Corsi, Trends Neurosci., 25:632-637, 2002.
Mulligan and Berg, Proc. Natl. Acad. Sci. USA, 78(4):2072-2076, 1981.
Murthy and Maklouf, J. Biol. Chem., 269:15977-15980, 1994.
Murthy et al., Ann. NY Acad. Sci., 805:355-362, 1996.
Neurath et al., Gut., 43(6):856-860, 1998.
O'Hare et al., Proc. Natl. Acad. Sci. USA, 78: 1527, 1981.
Okayama et al., Mol. Cell. Biol., 2(2):161-170, 1982.
Orihata and Sarna, Am. J. Physiol., 266:G665-676, 1994.
Orihata and Sarna, Dig. Dis. Sci., 41:1303-1309, 1996.
Orihata and Sarna, J. Pharmacol. Exp. Ther., 271:660-670, 1994.
Osborn et al., Proc. Natl. Acad. Sci. USA, 86:2336-2340, 1989.
Otterson and Sarna, Am. J. Physiol., 266:G576-584, 1994.
Palmer et al., Eur. J. Pharmacol. 132:163-170, 1986.
Pang et al., FEBS Lett., 546(2-3):349-354, 2003.
Parry and Mackman, J. Immunol., 159(11):5450-5456, 1997.
Patanjali et al., Proc. Natl. Acad. Sci. USA, 88(5):1943-1947, 1991.
Pazdrak et al., Gastroenterology, 127(4):1096-1109, 2004.
PCT Appln. WO 84/03564
PCT Appln. WO 98/07408
Perez-Reyes et al., Nature, 340(6230):233-236, 1989.
Pisegna and Wank, Proc. Natl. Acad. Sci. USA, 90(13):6345-6349, 1993.
Pluja et al., Eur. J. Pharmacol, 389(2-3):217-224, 2000.
Rao et al., Am. J. Physiol. Gastrointest. Liver Physiol., 280:G629-639, 2001.
Rawlings and Hezareh, Endocr. Rev., 17(1):4-29, 1996.
Remington's Pharmaceutical Sciences, 15^{th} ed., pages 1035-1038 and 1570-1580, Mack Publishing Company, Easton, Pa., 1980.
Russell et al., Mol. Cell. Biol., 14(4):2343-2351, 1994.
Ruth et al., Science, 245(4922):1115-1118, 1989.
Saada et al., Biochem. Biophys. Res. Commun., 302(1):23-28, 2003.
Sambrook et al., In: Molecular cloning, Cold Spring Harbor Laboratory Press, Cold Spring Harbor, N.Y., 2001.
Santerre et al., Gene, 30: 147-156, 1984.
Sarna and Daniel, Am. J. Physiol., 225:125-131, 1973.
Sarna and Daniel, Gastroenterology, 69:660-667, 1975.
Sarna and Shi, In: Function and regulation of colonic contractions in health and disease, San Diego, Calif., Elsevier, 2006.
Sarna et al., Am. J. Physiol., 257:G423-432, 1989.
Sarna, Am. J. Physiol. Gastrointest. Liver Physiol., 284:G789-797, 2003.
Sarna, Am. J. Physiol., 253:G697-705, 1987.
Sarna, Dig. Dis. Sci., 36(6):827-862, 1991.
Sarna, Dig. Dis. Sci., 36:827-862; 998-1018, 1991.
Sarna, Gastroenterology, 118:A837, 2000.
Sarna, In: In vivo myoelectric activity: methods, analysis, and interpretation, Bethesda, Am. Physiol. Soc., 1989.
Schaeffer and Weber, Mol. Cell. Biol., 19(4):2435-2444, 1999.
Schena, et al., Science, 270:467-470, 1995.
Schultzberg et al., Brain Res., 155(2):239-248, 1978.
Schwizer et al., Gut., 41:500-504, 1997.
Sethi and Sarna, Gastroenterology, 100(4):954-963, 1991.
Shalon et al., Genome Res., 6(7):639-645, 1996.
Shaywitz and Greenberg, Annu. Rev. Biochem., 68:821-861, 1999
Shi and Sarna, Am. J. Physiol. Gastrointest. Liver Physiol., 278:G234-242, 2000.
Shi and Sarna, Am. J. Physiol. Gastrointest. Liver Physiol., 286:G899-905, 2004.
Shi and Sarna, Gastroenterology, 2006.
Shi and Sarna, Gastroenterology, 112:864-874, 1997.
Shi et al., Electrophoresis, 24(19-20):3371-3377, 2003.
Shi et al., Gastroenterology, 129:1518-1532, 2005.
Shi et al., In: Human Colonic Circular Smooth Muscle Cells: A Novel Role in Regulation of Motility Function, Gastroenterology, 2006.
Shoemaker et al., Nature Genetics, 14:450-456, 1996.
Siebenlist et al., Annu. Rev. Cell Biol., 10:405-455, 1994.
Silva et al., Annu. Rev. Neurosci., 21:127-148, 1998.
Smith et al., Gene, 25(1):21-8, 1983.
Smyth-Templeton et al., DNA Cell Biol., 21(12):857-867, 1997.
Soldatov, Proc. Natl. Acad. Sci. USA, 89(10):4628-4632, 1992.
Somlyo and Somlyo, J. Physiol., 522(2):177-185, 2000.
Somlyo and Somlyo, Physiol. Rev., 83:1325-1358, 2003.
Sreedhaan et al., Biochem. Biophys. Res. Commun., 203(1):141-148, 1994.
Stewart and Young, In: Solid Phase Peptide Synthesis, 2d. ed., Pierce Chemical Co., 1984.
Summers et al., Gastroenterology, 70:753-758, 1976.
Svoboda et al., Biochem. Biophys. Res. Commun., 195(2):881-888, 1993.
Sweatt, J. Neurochem., 76(1):1-10, 2001.
Szybalski and Szybalski, Proc. Natl. Acad. Sci. USA, 48:2026-2034, 1962.
Takeyama et al., 1980.
Talmage and Mawe, J. Auton. Nerv. Syst., 43(1):83-89, 1993.
Tam et al., J. Am. Chem. Soc., 105:6442, 1983.
Tanabe et al., Nature, 328(6128):313-318, 1987.
Tsukamoto et al., Am. J. Physiol., 272:G1607-1614, 1997.
Uemura et al., Pediatr. Surg. Int., 14(1-2):66-70, 1998.
Vogt and Bos, Adv. Cancer Res., 55:1-35, 1990.
Wang et al., Regul. Pept., 84(1-3):55-67, 1999.
Weih et al., J. Immunol., 157:3974-3979, 1996.
Wigler et al., Cell, 11(1):223-232, 1977.
Wigler et al., Proc. Natl. Acad. Sci. USA, 77(6):3567-3570, 1980.
Wood, Am. J. Physiol., 222(1):118-125, 1972.
Wood, Gastroenterology. 127(2):635-657, 2004.
Wood, In: Physiology of the enteric nervous system, NY, Raven, 1994.
Xia et al., J. Pharmacol. Exp. Ther., 281(2):629-633, 1997.
Yamamoto et al., Nature, 334(6182):494-498, 1988.
Yao et al., Dig. Dis. Sci., 50:1569-1575, 2005.
Yin and Chen, Obes. Res., 13:1580-1587, 2005.
Zafirov et al., Eur. J. Pharmacol., 115(1):103-107, 1985.
Zhong, Nature, 375(6532):588-592, 1995.

1. A method for treating a gastrointestinal motility disorder in a subject comprising delivering to gut smooth muscle cells of the subject an effective amount of an α_{1C} modulator, whereby the modulator modulates the long-term expression of α_{1C} polypeptide in the cells.

2. (canceled)

3. The method of claim 1, wherein the α_{1C} modulator is administered to the subject for at least 3 hours.

4. (canceled)

5. The method of claim 1, wherein the α_{1C} modulator is formulated for time release or sustained release.

6. The method of claim 5, wherein the α_{1C} modulator is formulated for administration by transdermally, intravenously, orally, or intrarectally.

7. The method of claim 6, wherein the α_{1C} modulator is formulated as a patch or suppository.

8. The method of claim 3, wherein the subject is administered a low dosage of the α_{1C} modulator during or throughout the time period, wherein the low dosage is about or less than about 25 nM/kg/day.

9-12. (canceled)

13. The method of claim 1, wherein the subject has been diagnosed or is suspected of having constipation, irritable bowel syndrome, gastroparesis, or inflammatory bowel disease.

14. The method of claim 12, wherein the subject has been diagnosed or is suspected of having a gastrointestinal infection.

15. (canceled)

16. The method of claim 1, wherein the long-term expression of α_{1C} polypeptide is significantly increased.

17. The method of claim 16, wherein the α_{1C} modulator is an α_{1C} inducer.

18. (canceled)

19. (canceled)

20. The method of claim 17, wherein the α_{1C} inducer is selected from the group consisting of VIP, PACAP, an agonist of VIP or PACAP receptor, or norepinephrine.

21. The method of claim 20, wherein α_{1C} inducer is PACAP.

22. The method of claim 21, wherein PACAP is PACAP38.

23. The method of claim 20, wherein the α_{1C} inducer is VIP.

24. The method of claim 20, wherein the subject is administered more than one α_{1C} inducer selected from the group consisting of VIP, PACAP, norepinephrine, an agonist of VIP or PACAP receptor, or an agonist of a norepinephrine receptor.

25. The method of claim 16, wherein the α_{1C} modulator inhibits an α_{1C} transcription repressor.

26.-54. (canceled)

55. A method for suppressing contractility of gut smooth muscle cells in a subject comprising administering to the subject an effective amount of an α_{1C} repressor, whereby the effective amount represses the expression of α_{1C} polypeptide in the cells.

56.-59. (canceled)

60. The method of claim 55, wherein the subject has diarrhea.

61. (canceled)

62. A method for inducing sustained contractility or relaxation of smooth muscle cells in a subject comprising administering to the subject a relatively low dose of an α_{1C} modulator, whereby the modulator modulates the long-term expression of α_{1C} polypeptide in the cells.

63.-71. (canceled)


SEQUENCE LISTING (8 sequences)

SEQ ID NO: 1 - protein, 38 aa, Artificial Sequence
  Description of Artificial Sequence  Synthetic Peptide
HSDGIFTDSYSRYRKQMAVKKYLAAVLGKRYKQRVKNK

SEQ ID NO: 2 - protein, 27 aa, Artificial Sequence
  Description of Artificial Sequence  Synthetic Peptide
HSDGIFTDSYSRYRKQMAVKKYLAAVL

SEQ ID NO: 3 - protein, 28 aa, Artificial Sequence
  Description of Artificial Sequence  Synthetic Peptide
HSDAVFTDNYTRLRKQMAVKKYLNSILN

SEQ ID NO: 4 - DNA, 18 bp, Artificial Sequence
  Description of Artificial Sequence  Synthetic Primer
caccaaggttccaactat

SEQ ID NO: 5 - DNA, 25 bp, Artificial Sequence
  Description of Artificial Sequence  Synthetic Primer
ccatggtcaatgagaatacgaggat

SEQ ID NO: 6 - DNA, 20 bp, Artificial Sequence
  Description of Artificial Sequence  Synthetic Primer
gccgcattggcattcatgtt

SEQ ID NO: 7 - DNA, 19 bp, Artificial Sequence
  Description of Artificial Sequence  Synthetic Primer
cccatagttggaacacctc

SEQ ID NO: 8 - DNA, 25 bp, Artificial Sequence
  Description of Artificial Sequence  Synthetic Primer
gtgcatgaagctcaactcaactatt